(19) United States
(12) Patent Application Publication
(10) Pub. No.: US 2026/0207328 A1
(43) Pub. Date: Jul. 23, 2026

(54) SYSTEMS AND METHODS FOR TREATING VALVULAR REGURGITATION OF A DEFECTIVE CARDIAC VALVE

(71) Applicant: CroiValve Ltd., Dublin (IE)

(72) Inventors: Pascal LAUNOIS, Dublin (IE); Gavin KENNY, Galway (IE); Meadhbh PARSONS, Galway (IE); Ross SMITH, Covan (IE); Marianna PHILIPP, Dublin (IE)

(73) Assignee: CroiValve Ltd., Dublin (IE)

(21) Appl. No.: 19/454,205

(22) Filed: Jan. 20, 2026

Related U.S. Application Data
(60) Provisional application No. 63/747,869, filed on Jan. 21, 2025.

(51) Int. Cl. A61F 2/24; A61F 2/00

(52) U.S. Cl. CPC A61F 2/2418; A61F 2/0095; A61F 2/246; A61F 2/2463; A61F 2210/0014; A61F 2220/0008; A61F 2220/0025; A61F 2230/0006; A61F 2230/0067; A61F 2250/0003; A61F 2250/0018; A61F 2250/0036; A61F 2250/0039

(57) ABSTRACT

Apparatus and methods for repairing a cardiac valve, e.g., a tricuspid valve, are provided. The apparatus may include a prosthetic device coupled to an elongated support coupled to a stent configured to be implanted within a vessel, e.g., the superior vena cava, to suspend and maintain the prosthetic device within the cardiac valve. The support may include a proximal, delivery portion detachably coupled, in a delivery state, to a distal, implantable portion coupled to the prosthetic device. The prosthetic device may be formed of biocompatible material coupled to a frame, and may have prosthetic leaflets that allows blood to flow through in one direction during a phase of the cardiac cycle (e.g., diastole) but prevent blood regurgitation during the other phase (e.g., systole). The prosthetic device may include features configured to improve sealing at a regurgitant orifice of the cardiac valve.

### CROSS-REFERENCE TO RELATED APPLICATION

This application claims the benefit of priority of U.S. Provisional Patent Appl. No. 63/747,869, filed Jan. 21, 2025, the entire contents of which are incorporated herein by reference.

### TECHNICAL FIELD

This technology generally relates to systems and methods having improved sealing features for performing transcatheter or minimally invasive repair of a defective cardiac valve, such as the tricuspid, mitral, pulmonary, and aortic valves.

### BACKGROUND

The human heart has four major valves which moderate and direct blood flow in the cardiovascular system. These valves serve critical functions in assuring a unidirectional flow of an adequate blood supply through the cardiovascular system. The mitral valve and aortic valve control the flow of oxygen-rich blood from the lungs to the body. The mitral valve lies between the left atrium and left ventricle, while the aortic valve is situated between the left ventricle and the aorta. Together, the mitral and aortic valves ensure that oxygen-rich blood received from the lungs is ejected into systemic circulation. The tricuspid and pulmonary valves control the flow of oxygen-depleted blood from the body to the lungs. The tricuspid valve lies between the right atrium and right ventricle, while the pulmonary valve is situated between the right ventricle and the pulmonary artery. Together the tricuspid and pulmonary valves ensure unidirectional flow of oxygen-depleted blood received from the right atrium towards the lungs.

Heart valves are passive structures composed of leaflets that open and close in response to differential pressures on either side of the valve. The aortic, pulmonary, and tricuspid valves have three leaflets, while the mitral valve has only two leaflets. Dysfunction of the cardiac valves is common and can have profound clinical consequences. Regurgitation occurs when the valve leaflets do not meet, or “coapt” correctly, thus causing blood to leak backwards through the valve each time the heart pumps. Failure of the valves to prevent regurgitation leads to an increase in the pressure of blood in the lungs or liver and reduces forward blood flow, causing the heart to pump more blood to compensate for the loss of pressure. Such degradation may result in serious cardiovascular compromise or even death. Valvular dysfunction either results from a defect in the valve leaflet or supporting structure, or dilation of the fibrous ring supporting the valve. These factors lead to poor coaptation of valve leaflets, allowing blood to travel in the wrong direction.

Previously known medical treatments to address diseased valves generally involve either repairing the diseased native valve or replacing the native valve with a mechanical or biological valve prosthesis. Previously-known valve prostheses have some disadvantages, such as the need for long-term maintenance with blood thinners, the risk of clot formation, limited durability, etc. Accordingly, valve repair, when possible, usually is preferable to valve replacement. However, most dysfunctional valves are too diseased to be repaired using previously known methods and apparatus. Accordingly, a need exists for a prosthesis capable of assisting heart valve function that enables treatment of a larger patient population, while reducing the need to fully supplant the native heart valve.

For many years, the standard treatment for such valve dysfunction called for surgical repair or replacement of the valve during open-heart surgery, a procedure conducted under general anesthesia. An incision is made through the patient's sternum (sternotomy), and the heart is accessed and stopped while blood flow is rerouted through a heart-lung bypass machine. When replacing the valve, the native valve is excised and replaced with either a mechanical or biological prosthesis. However, these surgeries are prone to many complications and long hospital stays for recuperation.

More recently, transvascular techniques have been developed for introducing and implanting a replacement valve, using a flexible catheter in a manner less invasive than open-heart surgery. In such techniques, a replacement valve is mounted in a compressed state at the end of a flexible catheter and advanced through the blood vessel of a patient until the prosthetic valve reaches the implantation site. The valve then is expanded to its functional size at the site of the defective native valve, usually by inflating a balloon within where the valve has been mounted. By expanding the prosthetic valve, the native valve leaflets are generally pushed aside and rendered ineffective. Examples of such devices and techniques, wherein the native valve is replaced in its entirety by a substitute tissue valve, are described, for example, in U.S. Pat. Nos. 6,582,462 and 6,168,614 to Andersen.

Prostheses have been produced and used for over sixty years to treat cardiac disorders. They have been made from a variety of materials, both biological and artificial. Mechanical or artificial valves generally are made from non-biological materials, such as plastics or metals. Such materials, while durable, are prone to blood clotting and thrombus formation, which in turn increases the risk of embolization and stroke or ischemia. Anticoagulants may be taken to prevent blood clotting that may result in thromboembolic complications and catastrophic heart failure, however, such anti-clotting medication may complicate a patient's health due to the increased risk of hemorrhage.

In contrast, “bio-prosthetic” valves are constructed with prosthetic leaflets made of natural tissue, such as bovine, equine or porcine pericardial tissue, which functions very similarly to the leaflets of the natural human heart valve by imitating the natural action of the heart valve leaflets, coapting between adjacent tissue junctions known as commissures. The main advantage of valves made from natural tissue is they are not as prone to blood clots and do not absolutely require lifelong systemic anticoagulation.

In recent years, bio-prosthetic valves have been constructed by integrating prosthetic leaflets made from natural tissue into a stent-like supporting frame, which provides a dimensionally stable support structure for the prosthetic leaflets. In more advanced prosthetic heart valve designs, besides providing dimensionally stable support structure for the prosthetic leaflets, the stent-like supporting frame also imparts a certain degree of controlled flexibility, thereby reducing stress on the prosthetic leaflet tissue during valve opening and closure and extending the lifetime of the prosthetic leaflets. In most designs, the stent-like supporting frame is covered with a biocompatible cloth (usually a polyester material such as Dacron™ or polytetrafluoroethylene (PTFE)) that provides sewing attachment points for the prosthetic leaflet commissures and prosthetic leaflets themselves. Alternatively, a cloth-covered suture ring may be attached to the stent-like supporting frame, providing a site for sewing the valve structure in position within the patient's heart during a surgical valve replacement procedure.

While iterative improvements have been made on surgical bio-prosthetic valves over the last several decades, existing bio-prosthetic valves still have drawbacks. In most designs, the bio-prosthetic valve is implanted as a replacement for the native valve, filling the entire space the native valve had occupied. One drawback to this procedure is the mismatch in size and mass between opposing surfaces of the stent-like supporting frame. The mismatch is often due to the variability in the shapes and mechanical characteristics of the stent-like supporting frame. For prosthetic valves with balloon-expandable stent-like supporting frames, the recoil of the supporting frames post-balloon-inflation may lead to perivalvular leaks around the circumference of the prosthetic valve and potential slippage and migration of the valve post-implantation. Another risk associated with prosthetic valves having balloon-expandable supporting frames is potential damage to the prosthetic leaflets of the prosthesis during implantation, when the prosthetic leaflets may be compressed between the balloon and the supporting frame. For prosthetic valves with self-expanding stent-like supporting frames, mismatch may arise due to the deformation/movement of the supporting frame, e.g., slight deformation of the frame into a less than circular shape during normal cardiac movement. Such mismatch may lead to instability among components of a prosthetic valve, resulting in perivalvular leaks and uneven stress distribution in the prosthetic leaflets, resulting in accelerated wear of the valve.

Some innovation has addressed these problems by augmenting, rather than replacing, the native valve. The simplest of these devices is a plug suspended across the center of the valve that allows the native leaflets to coapt against the plug body to block regurgitation, as described in U.S. Pat. No. 7,854,762 to Speziali. Though the plug design helps to prevent regurgitation, the major drawback is that it also blocks some of the blood flow during diastole. Improved prostheses are described in U.S. Pat. Nos. 10,383,729 and 10,682,231 to Quinn, U.S. Pat. No. 10,952,854 to Heneghan et al., and U.S. Pat. No. 11,219,525 to Vesely et al., the entire contents of each of which are incorporated herein by reference.

It would be desirable to further enhance designs to, for example, allow easier delivery of a prosthetic device to a cardiac valve, provide a robust structure that ensures integrity of an implanted prosthetic including its prosthetic leaflets, improve coaptation of the device with the native leaflets to reduce regurgitation.

### SUMMARY

Provided herein are improved heart valve repair apparatus and methods that, for example, allow more reliable sealing of the prosthetic device at the native cardiac valve, provide robust structure, and minimize regurgitation. The apparatus and methods may be optimized for use in treating cardiac valve regurgitation when the native leaflets of the cardiac valve do not coapt correctly, thus causing blood to leak backwards through the valve as the heart pumps. Advantageously, apparatus may be configured for implantation at a cardiac valve within a blood flow path such that the native leaflets abut the apparatus during the portion of the cardiac cycle when the cardiac valve attempts to close, thereby enhancing native leaflet coaptation and minimizing regurgitation.

In accordance with one aspect, a coaptation assist valve device for placement across a patient's native heart valve having a regurgitant orifice is provided. The coaptation assist valve device may comprise a frame comprising a proximal structure, a distal structure, and a spine extending between the proximal and distal structures, a conduit comprising a proximal end coupled to the proximal structure, a distal end, and a flexible side wall defining a channel extending between the proximal and distal ends, the flexible side wall configured to transition between an inflated state, a contracted state, and an expanded coaptation state, and a plurality of prosthetic leaflets mounted within the channel of the conduit. At least a middle portion of the flexible side wall may have an outer diameter that is larger than an outer diameter of the proximal end in the inflated state. Moreover, an external surface of the middle portion of the flexible side wall may be configured to fill the regurgitant orifice and coapt with native leaflets of the native heart valve in the expanded coaptation state.

The plurality of prosthetic leaflets may be configured to allow diastolic blood flow therethrough, while preventing systolic blood flow therethrough. In addition, the plurality of prosthetic leaflets may be coupled to the proximal structure. For example, the proximal structure may comprise a plurality of prosthetic leaflet anchors configured to be coupled to the plurality of prosthetic leaflets. The frame may further comprise an inner structure coupled to the proximal structure via the plurality of prosthetic leaflet anchors. The inner structure may be configured to provide additional rigidity to the proximal structure. In some embodiments, the proximal structure and/or the distal structure may comprise a ring. For example, the ring may comprise a scallop, sinusoidal, or zig-zag shape. Moreover, the proximal structure may be coupled to the spine via a plurality of proximal spokes, and the distal structure may be coupled to the spine via a plurality of distal spokes. Additionally, the plurality of prosthetic leaflets may be formed from a same material as the flexible side wall.

In some embodiments, the distal end of the conduit may not be coupled to the distal structure. Accordingly, in the inflated state, the distal end of the conduit may have an outer diameter that is larger than an outer diameter of the distal structure. Moreover, the flexible side wall may be unsupported between the proximal and distal ends of the conduit. In the inflated state, at least a portion of the flexible side wall may have an outer diameter that increases in a direction from the proximal end towards the distal end. For example, in the inflated state, the flexible side wall may comprise a cone shape configured to reduce the fluid velocity and increase internal dynamic pressure of the conduit during diastole, to thereby reduce deflation of the flexible side wall. Additionally, the conduit may comprise a flexibility that varies along a length of the conduit, the flexibility configured to increase compliance of the conduit in a radial direction and increase stiffness of the conduit in an axial direction to prevent the conduit from prolapsing. For example, the conduit may comprise a plurality of stiffeners extending longitudinally between the proximal and distal ends of the conduit within the flexible side wall. The plurality of stiffeners may comprise a shape memory material pre-shaped to control an overall shape of the conduit.

Additionally, or alternatively, the flexible side wall may comprise a thickness that that varies along the length of the conduit. For example, the varying thickness of the flexible side wall may be defined by a number of layers of sheet material forming the flexible side wall along the length of the conduit. In some embodiments, a proximal region of the conduit may comprise a plurality of layers of sheet material configured to increase rigidity and maintain the shape of the proximal region of the conduit. Additionally, or alternatively, the flexible side wall may comprise an outer layer of sheet material comprising a plurality of outer panels, and an inner layer of sheet material comprising a plurality of inner panels. The plurality of outer panels and inner panels may be sutured together to define a plurality of longitudinally extending support zones configured to increase axial rigidity of the conduit, e.g., to control the shape of the conduit during valve crossing, prevent backfolding of the conduit over the proximal portion of the frame, and/or prevent excessive deflation in combination with the Bernoulli principal, as described in further detail below, and expose blood flow across the flexible side wall to a smooth side of sheet material to reduce shear stress on the blood. The plurality of longitudinally extending support zones may be disposed along a circumference of the flexible side wall.

A first longitudinally extending support zone of the plurality of longitudinally extending support zones may comprise a first outer panel of the plurality of outer panels of the outer layer sutured to folded portions of adjacent inner panels of the plurality of inner panels of the inner layer, and the adjacent inner panels may be sutured together. The folded portions of the adjacent inner panels may be folded towards the outer layer of sheet material. Additionally, a second longitudinally extending support zone of the plurality of longitudinally extending support zones may comprise a first inner panel of the plurality of inner panels of the inner layer sutured to overlapping portions of adjacent outer panels of the plurality of outer panels of the outer layer. In some embodiments, the varying thickness of the flexible side wall may be defined by at least one thick longitudinally extending strip of sheet material sutured in a manner to define a hinge extending along the length of the conduit, the hinge configured to function as a folding apex configured to be aligned with a commissure of the native heart valve to promote deeper sealing of the regurgitant orifice. Additionally, or alternatively, the flexible side wall may be pre-formed to comprise weak portions along the length of the conduit to vary the flexibility of the conduit along the length of the conduit.

In the inflated state, the distal end of the conduit may extend a predetermined distance distally beyond the distal structure. Moreover, the conduit may comprise a plurality of flexible tethers, each having a first end coupled to the distal structure and a second end coupled to an inner surface of the middle portion of the flexible side wall. The plurality of flexible tethers may be configured to prevent prolapse of the flexible side wall over the frame. For example, the plurality of flexible tethers may be formed of a suture material and/or a plurality of pericardium strips. Further, each of the plurality of flexible tethers may have a length selected to control an overall shape of the conduit. In some embodiments, the overall shape of the conduit may comprise an oval shape. Accordingly, the conduit may be configured to be positioned relative to the native heart valve such that a long axis of the oval shape is aligned with the regurgitant orifice. Additionally, in the expanded coaptation state, the plurality of flexible tethers may be configured to be tensioned to permit the flexible side wall to fill the regurgitant orifice and coapt with native leaflets of the native heart valve.

In some embodiments, the distal end of the conduit may be coupled to the distal structure, such that, in the inflated state, a proximal portion of the conduit may have a cross-sectional area that increases from the proximal end towards the middle portion, and a distal portion of the conduit may have a cross-sectional area that increases from the distal end towards the middle portion. In addition, the flexible side wall may define a plurality of windows disposed on at least the distal portion of the conduit, the plurality of windows sized and shaped to permit blood flow therethrough to promote inflation of the flexible side wall during systole. Moreover, unsupported edges of the plurality of windows may be configured to facilitate compliance of the flexible side wall to the regurgitant orifice. For example, blood flow through the plurality of windows may be configured to reduce the fluid velocity and increase internal dynamic pressure of the conduit during diastole, to thereby reduce deflation of the flexible side wall. In some embodiments, in the inflated state, the middle portion of the conduit may have an outer diameter that increases in a direction from the proximal end towards the distal end.

Further, during systole, an atrial side of the conduit proximal to a native leaflet sealing plane of the conduit may be configured to inflate to an outer diameter greater than a sealing contact between the conduit and the native leaflets at the native leaflet sealing plane. In addition, an internal pressure of the conduit decreases from systole to diastole, such that, during the patient's cardiac cycle, radial motion of the conduit may be generated to promote blood washout and reduce risks associated with blood stasis. A ratio of a width of the plurality of windows and a length of the unsupported edges of the plurality of windows may be selected to prevent the plurality of windows from folding upon release of an external force on the distal portion of the conduit. In some embodiments, in the inflated state, an inner surface of the proximal portion of the conduit may be separated from the proximal structure coupled to the plurality of prosthetic leaflets by a gap, the gap configured to promote blood washout and reduce risks associated with blood stasis. The flexible side wall may be unsupported between the proximal and distal ends of the conduit.

Alternatively, in some embodiments, the frame may comprise a plurality of support strips extending longitudinally between the proximal and distal structures, the plurality of support strips configured to support the flexible side wall in the contracted state to prevent the flexible side wall from collapsing onto the spine. In some embodiments, the conduit may comprise a proximal support extending circumferentially along at least a proximal region of the flexible side wall, a distal support extending circumferentially along at least a distal region of the flexible side wall, and/or one or more longitudinal supports extending longitudinally between the proximal and distal supports, the proximal, distal, and/or longitudinal supports configured to prevent prolapse of the flexible side wall over the frame. Additionally, or alternatively, the conduit may comprise a plurality of internal supports extending longitudinally between the proximal and distal ends of the conduit, the plurality of internal supports configured to prevent prolapse of the flexible side wall over the frame.

In some embodiments, the frame may comprise a second proximal structure adjacent the proximal structure. For example, the second proximal structure may be configured to be coupled to the middle portion of the flexible side wall to prevent prolapse of the flexible side wall over the frame. The second proximal structure may have an outer diameter that is larger than an outer diameter of the proximal structure. Moreover, the second proximal structure may comprise a ring that is coupled to the spine via a plurality of proximal spokes. Further, the conduit may comprise a plurality of flexible tethers, each having a first end coupled to the distal structure and a second end coupled to an inner surface of the middle portion of the flexible side wall, the plurality of flexible tethers configured to control the shape of the conduit in the inflated state and prevent prolapse of the flexible side wall over the frame.

The coaptation assist valve device further may comprise a support coupled to the spine, the support configured to suspend the conduit across the native heart valve. Moreover, the support may comprise a stent configured to engage the wall of a blood vessel. Accordingly, the stent may be configured to retain the support in a position in the patient to maintain the conduit in position across the native heart valve. Additionally, the support may be configured to suspend the conduit across the native heart valve without anchoring of the coaptation assist valve device to an annulus of the native heart valve or atrial or ventricular tissue adjacent to the native heart valve.

In accordance with another aspect, a holder device is provided for supporting a coaptation assist valve device during storage within a container, the coaptation assist valve device comprising a proximal structure, a distal structure, a spine extending between the proximal and distal structures, and a flexible conduit having a proximal end coupled to the proximal structure and an at least partially unsupported distal end. The holder device may comprise a base sized and shape to minimize movement between the holder device and the container when the holder device is disposed within the container, a base sized and shape to minimize movement between the holder device and the container when the holder device is disposed within the container, and a spine support comprising a plurality of extensions extending laterally from the base, the plurality of extensions configured to be inserted through the distal structure and defining a lumen configured to receive the spine of the coaptation assist valve device. Accordingly, when the spine of the coaptation assist valve device is received within the lumen and the holder device is disposed within the container, the holder device may be configured to support the shape of flexible conduit in an expanded state to thereby prevent folding of the at least partially unsupported distal end of the flexible conduit.

The base may comprise a geometry corresponding to a geometry of an internal cavity of the container. For example, the base may comprise an arcuate shape. Moreover, the plurality of extensions of the spine support may be biased towards a radially inward collapsed state to thereby apply a force against the spine when the spine is received within the lumen. When at least a portion of the distal end of the flexible conduit is coupled to the distal structure of the coaptation assist valve device to define a plurality of windows, the holder device further may comprise a plurality of prongs extending laterally from the base, the plurality of prongs sized and shaped to be inserted through the plurality of windows. Accordingly, when the plurality of prongs are disposed within the flexible conduit via the plurality of windows and the holder device is disposed within the container, the plurality of prongs may be configured to support the plurality of windows and the flexible conduit in the expanded state. In addition, each of the plurality of prongs may comprise a pair of fingers, the pair of fingers configured to transition between a collapsed configuration where the pair of fingers are sized and shaped to be inserted through the plurality of windows, and an expanded configuration where the pair of fingers are configured to support the flexible conduit in the expanded state. The pair of fingers may be biased towards the expanded state. Additionally, the plurality of prongs may comprise a number of prongs corresponding with a number of windows of the plurality of windows of the coaptation assist valve device.

### BRIEF DESCRIPTION OF THE DRAWINGS

FIG. 1 is a perspective view of an exemplary heart valve therapeutic device for repairing a defective heart valve.

FIG. 2 illustrates exemplary detachable supports constructed in accordance with some embodiments.

FIGS. 3A and 3B are perspective views of an exemplary prosthetic device of the heart valve therapeutic device of FIG. 1.

FIG. 3C is a cross-sectional view of the prosthetic device of FIGS. 3A and 3B.

FIGS. 3D and 3E are perspective views of an exemplary frame of the prosthetic device of FIGS. 3A-3C.

FIG. 3F is a close up view of an exemplary step up of the frame of the prosthetic device of FIGS. 3A-3C.

FIG. 4A illustrates the implantable portion of the heart valve therapeutic device of FIG. 1 implanted at a native heart valve.

FIGS. 4B and 4C illustrate the heart valve therapeutic device at a native heart valve in a closed and open configuration, respectively.

FIGS. 5A to 5H are views of an exemplary method for introducing the prosthetic device of the heart valve therapeutic device across a native heart valve for implantation.

FIGS. 6A and 6B illustrate the implanted prosthetic device suspended across a native heart valve.

FIG. 7 illustrates various types of tricuspid regurgitation.

FIGS. 8A and 8B illustrate an alternative exemplary prosthetic device of the heart valve therapeutic device having a flared distal end constructed in accordance with some embodiments.

FIG. 9 illustrates an alternative exemplary prosthetic device having a flared distal end and reinforcement skirt shaping elements constructed in accordance with some embodiments.

FIGS. 10A and 10B illustrate an alternative exemplary prosthetic device having a flared distal end and a plurality of distal ring tethers constructed in accordance with some embodiments.

FIGS. 11A to 11H illustrate another alternative exemplary prosthetic device having a flared distal end and a plurality of distal ring tethers constructed in accordance with some embodiments.

FIGS. 12A and 12B illustrate an alternative exemplary prosthetic device having an enlarged middle region constructed in accordance with some embodiments.

FIGS. 13A to 13E illustrate an alternative exemplary prosthetic device having an enlarged middle region and a plurality of distal windows constructed in accordance with some embodiments.

FIGS. 14A to 14C illustrate an alternative exemplary prosthetic device having an enlarged middle region, distal windows, and reinforcement skirt support elements constructed in accordance with some embodiments.

FIG. 15 illustrates an alternative exemplary prosthetic device having an enlarged middle region, distal windows, and circumferential and longitudinal reinforcement skirt support elements constructed in accordance with some embodiments.

FIG. 16 illustrates an alternative exemplary prosthetic device having an enlarged middle region, distal windows, and longitudinal reinforcement skirt support elements constructed in accordance with some embodiments.

FIGS. 17A and 17B illustrate an alternative exemplary prosthetic device having an enlarged middle region, distal windows, and an additional proximal reinforcement frame constructed in accordance with some embodiments.

FIGS. 18A and 18B illustrate an alternative exemplary prosthetic device having an enlarged middle region, distal windows, and a plurality of distal ring tethers constructed in accordance with some embodiments.

FIGS. 19A to 19C illustrate an alternative exemplary prosthetic device having an enlarged middle region, distal windows, and a plurality of overlapping layers of pericardium constructed in accordance with some embodiments.

FIGS. 20A and 20B illustrate an exemplary prosthetic device holder constructed in accordance with some embodiments.

### DETAILED DESCRIPTION

Embodiments of this technology are directed to exemplary systems and methods for reducing cardiac valve regurgitation. Provided herein is a prosthetic device that may contain a prosthetic coaptation body to be positioned at a native cardiac valve. The prosthetic device may be suspended across the native heart valve by a support. For example, the support may be coupled to the prosthetic coaptation body and extend out of the heart into an adjacent blood vessel coupled to the heart (e.g., superior vena cava, inferior vena cava). The support may be coupled to the blood vessel with an anchor that preferably is expandable and has a stent structure. In some examples, the support is structured to suspend the prosthetic coaptation body in the native valve in a free-standing manner without anchoring to cardiac tissue, thereby minimizing damage to the heart. The prosthetic coaptation body may be formed from a frame (e.g., metal frame such as Nitinol) that is at least partially covered by a skirt made from biocompatible material, and also includes prosthetic leaflets. The frame, biocompatible material, and prosthetic leaflets may together form a conduit through which blood flows when the prosthetic leaflets open during the cardiac cycle.

The design of the prosthetic device improves coaptation with the native heart valve leaflets and allows for a more reliable delivery. The prosthetic device may be implanted percutaneously via a blood vessel, e.g., the jugular vein, femoral vein, femoral artery, for the treatment of a defective cardiac valve, e.g., tricuspid, mitral, pulmonary, or aortic valve. In one example, the prosthetic device may be used to treat symptomatic primary or functional (secondary) tricuspid regurgitation. For example, the prosthetic device may be positioned between the native tricuspid valve leaflets to restore the valve function without altering the native anatomy or obstructing flow during diastole and held in place by an anchor system deployed in an anchor site, e.g., within the heart and/or within a blood vessel coupled to the heart such as the superior vena cava (SVC).

The frame may be designed with predefined kink points or collapsible/expandable features to allow the conduit to be compressed into a delivery sheath without being damaged, and to more reliably expand upon delivery. The frame may have a proximal ring and a distal ring, as well as an inner ring coupled to the proximal ring via a plurality of skirt anchors to which the prosthetic valve leaflets may be attached. One or more of the rings may exhibit a scallop, sinusoidal, zig-zag shape or otherwise oscillating pattern in the expanded state to further improve the compression and expansion of the frame. The skirt of the prosthetic coaptation body may join the proximal ring to the distal ring to improve coaptation of the native valve against the skirt. The prosthetic coaptation body may be coupled to the support by a plurality of tethers/spokes that may be formed of shape-memory material such as Nitinol. The tethers/spokes may be rigid or stiff and hold the prosthetic coaptation body in position more accurately than tensile wires.

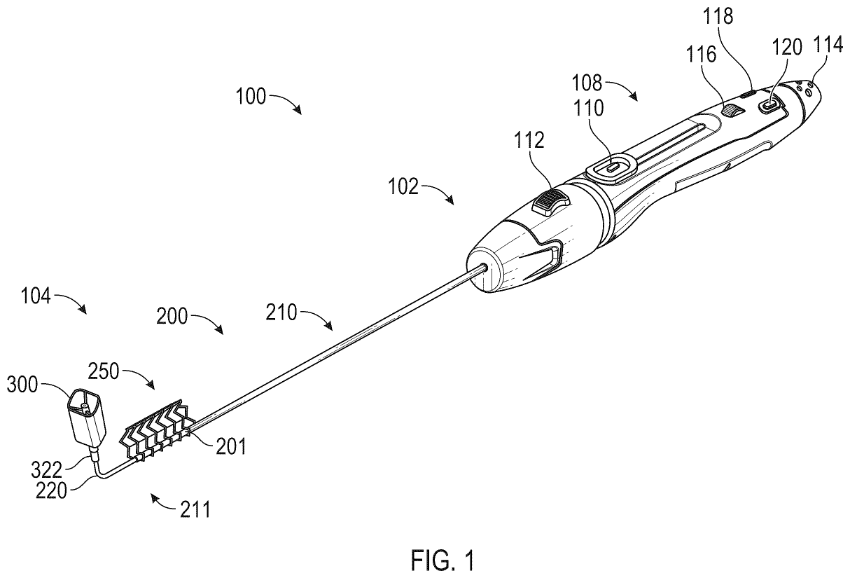

Referring to FIG. 1, an illustrative embodiment of exemplary heart valve therapeutic device 100 is described. Illustratively, heart valve therapeutic device 100 is designed for repairing a defective tricuspid valve. As will be understood by a person having ordinary skill in the art, heart valve therapeutic device 100 may be readily adapted for other cardiac valves such as the mitral valve, aortic valve, or pulmonary valve. Heart valve therapeutic device 100 may be constructed as described in Int'l Patent Appl. Pub. No. WO 2024/161309 A1, Int'l Patent Appl. Pub. No. WO 2023/026113 A1, and U.S. Pat. No. 11,219,525, the entire contents of each of which are incorporated by reference herein.

As illustrated in FIG. 1, heart valve therapeutic device 100 may include prosthetic device 300 coupled to support 200 at distal region 104 of heart valve therapeutic device 100, as well as actuator 108 at proximal region 102 of heart valve therapeutic device 100. Actuator 108 may include one or more handles configured to be manipulated by a clinician to deliver the system for implantation. Support 200 may include an elongated shaft including proximal, delivery portion 210 and distal, implantable portion 211, such that proximal, delivery portion 210 is removeably coupled to distal, implantable portion 211 at detachment area 201, and distal, implantable portion 211 is coupled to prosthetic device 300 at valve connection area 322. Heart valve therapeutic device 100 is structured to deliver prosthetic device 300 to a damaged native heart valve for an acute or chronic treatment, and certain components of heart valve therapeutic device 100 such as distal, implantable portion 211 of support 200 may be designed to be fully implanted long-term for the chronic treatment.

Distal, implantable portion 211 of support 200 further may include anchor 250. Anchor 250 may be formed of a stent structure and is preferably collapsible in a contracted, delivery state and expandable to an expanded, deployed state to anchor the prosthetic device at the native cardiac valve. For example, anchor 250 may contact the inner wall of a blood vessel (e.g., the SVC or IVC) to anchor distal, implantable portion 211 of support 200 intraluminally, thereby anchoring prosthetic device 300 in a free-standing, suspended manner in the native heart valve. As shown in FIG. 1, anchor 250 may be positioned on distal, implantable portion 211 adjacent to detachment area 201 of support 200. In some examples, detachment area 201 is located within anchor 250 such that the proximal end of anchor 250 provides the proximal-most position of the implantable portion of the device.

Actuator 108 is designed to be held and manipulated by a clinician and may include one or more interfaces such as interfaces 110, 112, 114, 116, 118, and 120. As illustrated, actuator 108 may be coupled to the proximal region support 200, and interfaces 110, 112, 114, 116, 118, and 120 may each be coupled to corresponding components of support 200 such that actuation of the interfaces cause movements described herein for delivery and implantation of prosthetic device 300, as described in U.S. Pat. No. 11,219,525. For example, interfaces 110, 112, 114, 116, 118, and 120 may be buttons, sliders, knobs, or the like that are actuated to deliver prosthetic device 300, manipulate support 200 for suitable implantation, lock distal components of distal, implantable portion 211 together, and/or to detach proximal, delivery portion 210 from distal, implantable portion 211. Accordingly, responsive to actuation of the interfaces of actuator 108, prosthetic device 300 may be manipulated for suitable positioning within the target native heart valve, the distal components of distal, implantable portion 211 may be locked together, and proximal, delivery portion 210 may be detached from distal, implantable portion 211.

For example, interface 110 may be operatively coupled to the shaping catheter for making extension adjustments to extend prosthetic device 300 into implantation position. Interface 112 may be operatively coupled to the elongated rail of support 200 for adjusting the angle of the rail for positing the prosthetic device 300 at the appropriate angle relative to the native heart valve. Interface 114 may be operatively coupled to the body support catheter for telescoping adjustments to extend or retract prosthetic device 300 to the native heart valve. Interface 116 may be operatively coupled to a first lock to lock distal, implantable components of the support together for implantation. For example, interface 116 may be operatively coupled to the body support catheter pusher for actuating the body support catheter lock. Interface 118 may be operatively coupled to a second lock to lock different distal, implantable components of the support together for implantation, such as locking to the anchor system. For example, interface 118 may be operatively coupled to the shaping catheter pusher for actuating the shaping catheter lock. Interface 120 may be operatively coupled to the anchor tube sleeve for disengaging the anchor tube.

In some configurations, an interface, e.g., interface 116, 118, may be moved distally along handle to cause portions of support 200 to move distally in a corresponding manner to facilitate locking of the distal components of distal, implantable portion 211 to secure the components in the implantable, locked position suitable for short-term (acute) or long-term (chronic) implantation of prosthetic device 300 at the native cardiac valve. Further, the same or different interface(s) may be moved proximally along the handle to cause detachment of the proximal, delivery portion 210 from distal, implantable portion 211 such that proximal, delivery portion 210 may be removed from the patient while distal, implantable portion 211 remains implanted, as explained in detail below. Interfaces 110, 112, 114, 116, 118, and 120 may be manually operated or controlled remotely using motorized controls, and actuator 108 may be actuated to reattach proximal, delivery portion 210 to distal, implantable portion 211 post-implantation in a follow-up procedure to permit adjustments after implantation of prosthetic device 300.

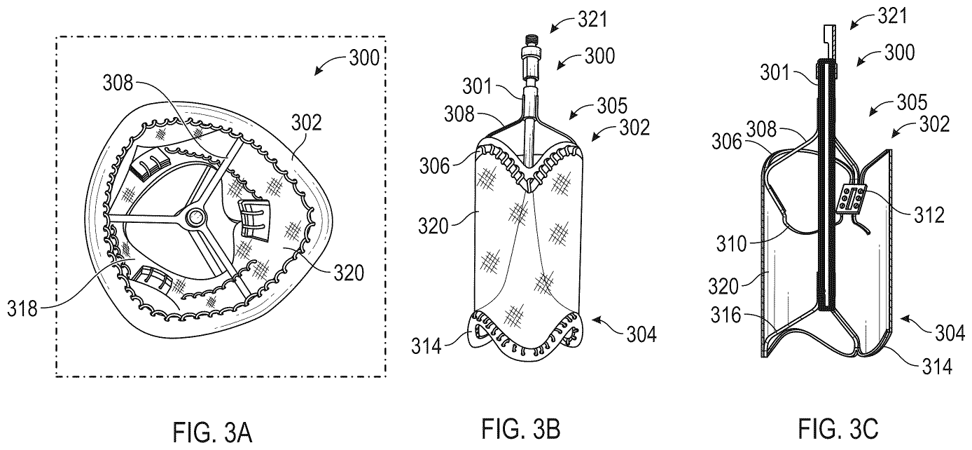

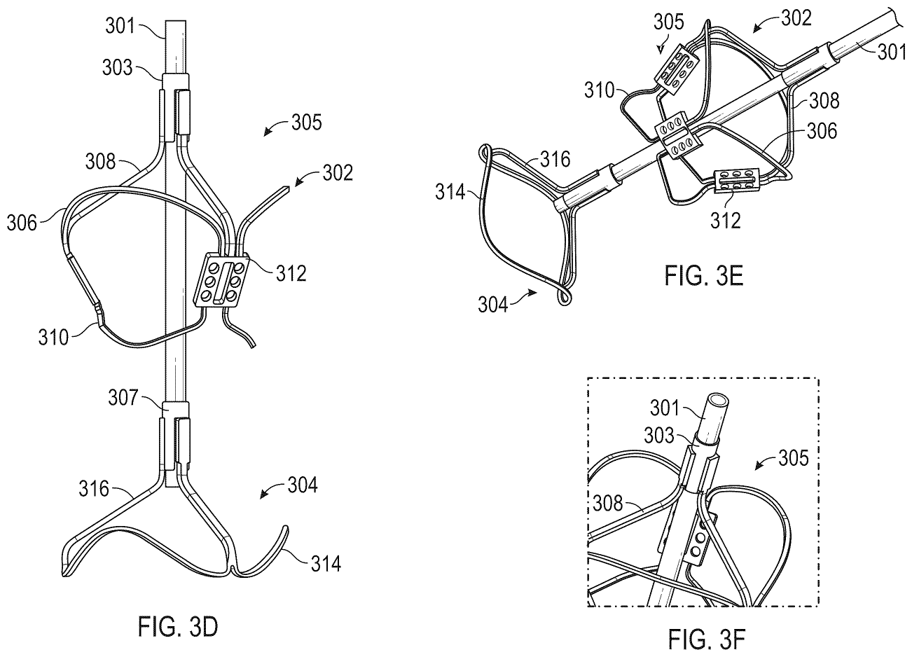

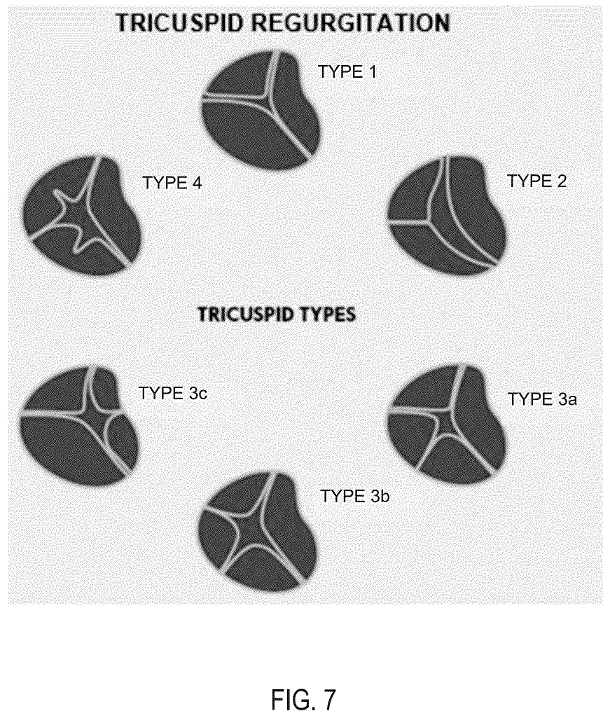

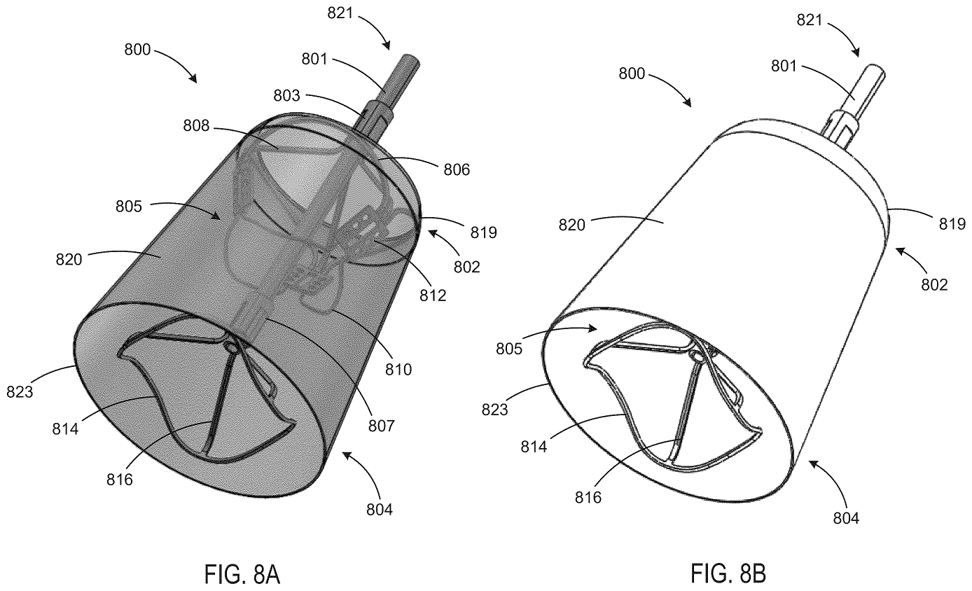

Prosthetic device 300 may be a prosthetic coaptation body 300, as illustrated, that includes a prosthetic valve structured to enhance the function of the native heart valve, which is described in further detail with regard to FIGS. 3A, 3B, and 3C below. Preferably, prosthetic coaptation body 300 works together with the native leaflets to both provide a surface for the native leaflets to coapt and to provide a prosthetic valve in a conduit formed by prosthetic coaptation body 300. Unlike prior prosthetic valves that do not use the native leaflets (e.g., because they are cut away or pushed aside by the implant), prosthetic coaptation body 300 may use both the native leaflets and the prosthetic leaflets in the same native heart valve, thereby creating a “double-valve” configuration in the single heart valve. As shown in FIG. 1, support 200 may be structured to suspend and maintain prosthetic coaptation body 300 across the native heart valve once it has been positioned appropriately. Alternatively, instead of prosthetic coaptation body 300, the prosthetic device may be any of the prosthetic coaptation bodies described below with regard to FIGS. 8A to 19C. As will be understood by one skilled in the art, the illustrated prosthetic coaptation body 300 may be substituted for other prosthetic devices designed to be implanted at a cardiac valve such as a plug/spacer device that coapts with native leaflets to reduce regurgitation such as that shown in U.S. Pat. No. 7,854,762 to Speziali.

As described above, distal, implantable portion 211 of support 200 may be coupled to prosthetic coaptation body 300. Proximal, delivery portion 210 of support 200, may be operatively coupled to actuator 108 and removeably coupled to distal, implantable portion 211 during delivery, such that proximal, delivery portion 210 may be manipulated by actuator 108 to accurately position prosthetic coaptation body 300 across the native valve. Support 200 may have a predefined bend to improve positioning of prosthetic coaptation body 300 across the native valve, as described in U.S. Pat. No. 11,219,525. For example, the bend may be predefined for a specific patient anatomy. Moreover, the predefine bend permits steering of the support from the predefined shape, which may have the effect of reducing stresses and strain on the elongated rail for long-term implant. In addition, heart valve therapeutic device 100 may include one or more radiopaque markers for in-vivo visualization during delivery of prosthetic coaptation body 300.

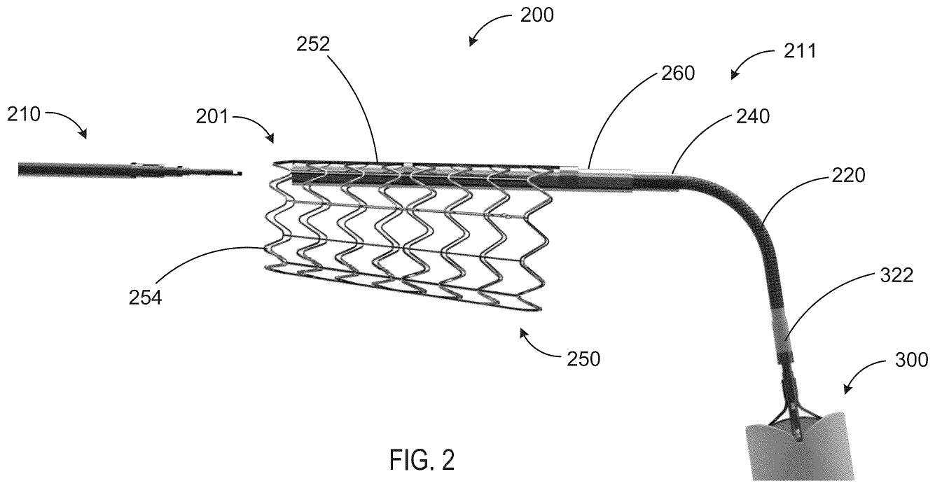

Referring now to FIG. 2, support 200 for delivering and implanting the prosthetic coaptation body is described. Support 200 includes an elongated shaft including proximal, delivery portion 210 and distal, implantable portion 211, and may be steered and manipulated via actuator 108 (see FIG. 1). Proximal, delivery portion 210 may be removeably coupled to distal, implantable portion 211 at detachment area 201 during delivery of prosthetic coaptation body 300, and proximal, delivery portion 210 may be decoupled from distal, implantable portion 211 to thereby implant distal, implantable portion 211 and prosthetic coaptation body 300 in the desired location within the patient. Accordingly, proximal, delivery portion 210 may be removed from the patient, while distal, implantable portion 211 and prosthetic coaptation body 300 remain fully implanted. As shown in FIG. 2, distal, implantable portion 211 of support 200 includes valve connection area 322 for coupling with prosthetic coaptation body 300 (e.g., via spine connector 321), anchor 250 including stent 254 coupled to anchor tube 260 via anchor support 252, and a plurality of catheters including body support catheter 220 and shaping catheter 240 for maneuvering and implanting prosthetic coaptation body 300, as described in U.S. Pat. No. 11,219,525. For example, anchor tube 260 may have a lumen sized and shaped to receive shaping catheter 240 therethrough, and may be coupled to one side of stent 254 to stabilize distal, implantable portion 211 of support 200 and bias it to one side of the blood vessel.

As described above, the elongated rail may extend from actuator 108 to prosthetic coaptation body 300, and may have a pre-formed bend area to facilitate delivery of prosthetic coaptation body 300 to the native heart valve. In addition, the pre-formed bend may reduce the stress required to position prosthetic coaptation body 300 during delivery. For example, the elongated rail may be an elongated shaft made of metal (e.g., Nitinol) that is preformed to a predetermined angle (e.g., 50-150 degree bend, 100 degree bend). Body support catheter 220 may be coaxial to the preformed rail and shaping catheter 240 and may be attached to prosthetic device 300 to facilitate telescoping of the prosthetic device beyond the bend. Shaping catheter 240 may be coaxial to the preformed rail and body support catheter 220. In some embodiments, the distal end of shaping catheter 240 may have a collar that is used to bend and straighten the preformed rail based on the relative axial position between the two responsive to actuation at the handle. For example, as shaping catheter 240 is advanced distally over the preformed bend of the elongated rail, the elongated rail straightens, and as shaping catheter 240 is retracted proximally relative to the elongated rail, the elongated rail returns to its natural state with the preformed bend. As will be understood by a person having ordinary skill in the art, the rail may be moved while the shaping catheter remains stationary within the patient to bend and straighten the preformed rail. In some embodiments, support 200 may have a plurality of pre-formed bends, e.g., two pre-formed bends, to facilitate positioning of prosthetic coaptation body 300 across the native valve, as described in WO 2024/161309 A1.

The prosthetic device may be secured on the distal end of the anchor system using the implantable support catheter, e.g., support 200, connected to a Nitinol Stent with a disconnectable proximal section to support delivery. The support catheter may be used to deliver and adjust and finally stabilize the position the prosthetic device across the native cardiac valve. The anchor system may be used to deploy, position, and support the prosthetic device, attached to the distal end. Once the prosthetic device has been positioned, a self-expanding Nitinol stent, e.g., stent 254, may be deployed in the tissue (e.g., SVC or IVC) which may be attached to the support catheter. The position of the prosthetic device may be further adjusted after the stent is deployed. As shown in FIG. 2, stent 254 may be supported by anchor tube 260 of support 200. The support catheter may have a steerable distal portion that is controlled by the handle to ensure optimal positioning. This can avoid hooks, screws or clamps in the thin, frail structures of the dilated right heart and allows the system to accommodate cardiac and respiratory motion. After deployment and final positioning of the prosthetic device, the position is locked and the delivery section of the anchor system, proximal to the stent, e.g., proximal, delivery portion 210, is disconnected and removed.

Advantageously, the anchor system allows for deflection from a straight configuration through 100 degrees of angulation so that the prosthetic device can be positioned coaxial to the tricuspid annulus; telescoping of the prosthetic device down, towards the apex of the ventricle, into the tricuspid annulus so that it is positioned properly between the tricuspid valve leaflets; extension and rotation of the position of the bend relative to the stent so that the prosthetic device can cross the tricuspid valve perpendicular to it, and so the clinician may freely position the stent to a preferred location; stabilizing of the prosthetic device in position by anchoring against the tissue (e.g., anchoring anchor 250 against the wall of a blood vessel such as the SVC or IVC); fixing the selected position, angulation and telescoping, of the prosthetic device. The positioning of the prosthetic device is helped by the native valve leaflets which naturally direct and center it within the central gap of the leaflets. The distal portion of the anchor system, e.g., distal, implantable portion 211, has sufficient stiffness to maintain the prosthetic device in position during the cardiac cycle, as well as sufficient flexibility to permit the prosthetic device to “self-center” within the native valve during systole. This distal portion of the anchor system may be connected to support the prosthetic device and stabilized in the SVC by the stent anchor. The sterilization process for the anchor system and its accessories may be Ethylene Oxide (ETO) or radiation sterilized. This sterilization process is standard for catheter systems.

Referring now to FIGS. 3A, 3B and 3C, exemplary prosthetic coaptation body 300 of heart valve therapeutic device 100 is described. FIG. 3A shows prosthetic coaptation body 300 viewed from the distal end downward, FIG. 3B is a side view of prosthetic coaptation body 300, and FIG. 3C is a cross-sectional view of prosthetic coaptation body 300. Prosthetic coaptation body 300 preferably includes frame 305 having prosthetic leaflets 318 coupled thereto. Prosthetic leaflets 318 may be formed from natural tissue, such as bovine, equine, or porcine pericardial tissue, and/or manmade, synthetic material suitable for implantation such as ePTFE. Prosthetic coaptation body 300 may also contain one or more biocompatible materials, e.g., formed from the natural tissue and/or manmade material, coupled to frame 305 such as skirt 320. Prosthetic leaflets 318 and skirt 320 may be formed from the same material and may be integrally formed from a common piece of material or may be separate. Frame 305 further may include spine connector 321 for coupling with a support connector of support 200 as described in further detail below. Moreover, the shape of prosthetic coaptation body 300 may be formed by frame 305, which is designed to transition from a contracted, delivery state to an expanded, deployed state and may be formed from shape memory material such as Nitinol. For example, frame 305 may form a conduit that, together with prosthetic leaflets 318 and the biocompatible material covering form a channel to allow blood to travel through prosthetic leaflets 318, when opened during the cardiac cycle, and through prosthetic coaptation body 300.

Skirt 320 may be a thin sheet of biocompatible material surrounding frame 305, extending from proximal ring 306 to distal ring 314 to form the outside surface of the conduit to which the native leaflets coapt when closed during the cardiac cycle. For example, skirt 320 may be sewn to proximal ring 306 and distal ring 314. Skirt 320 may be made of a rigid or compliant material. In some examples, skirt 320 expands and contracts responsive to pressure changes during the cardiac cycle. In this manner, skirt 320 may provide better coaptation with native leaflets. Accordingly, as prosthetic coaptation body 300 sits between the native tricuspid valve leaflets, it fills the regurgitant orifice area caused by right ventricular dilation. The native tricuspid valve leaflets seal against skirt 320 to prevent regurgitation between the native leaflets and prosthetic coaptation body 300 during systole. In addition, prosthetic leaflets 318 integrated within prosthetic coaptation body 300 supports flow during diastole. Prosthetic leaflets 318 may coapt onto the valve frame spine during systole to reduce regurgitation.

Referring now to FIGS. 3D, 3E, and 3F, frame 305 is described. Frame 305 may be made of metal, such as Nitinol or stainless steel. Frame 305 may be made of various components including wires, tubes, or flat strips. In a preferred embodiment, frame 305 is laser cut from one or more tubes of Nitinol. Frame 305 preferably includes spine 301, proximal portion 302 having proximal ring 306 and inner ring 310, and distal portion 304 having distal ring 314. Proximal ring 306 may be coupled to spine 301 via a plurality of proximal tethers 308 and step 303, as described in further detail below with regard to FIG. 3F, and distal ring 314 may be coupled to spine 301 via a plurality of distal tethers 316 and step 307. Alternatively, proximal tethers 308 and distal tethers 316 may be coupled directly to spine 301 without step 303 and step 307, respectively. Spine 301 may be an elongated shaft, e.g., formed from a metal tube such as stainless steel or Nitinol. As shown, spine 301 may extend generally through the middle of prosthetic coaptation body 300 to provide strength and support.

In the illustrated example, spine 301 extends through prosthetic coaptation body 300 to the connection with distal ring 314 and proximally past the proximal end of prosthetic coaptation body to permit permanent, secure coupling between spine 301 and support 200. Preferably, proximal tethers 308 and distal tethers 316 are formed of a rigid structure, and are compressible for delivery and may be self-expandable. Inner ring 310 may be positioned distal to and provide additional support to proximal ring 306, and may be coupled to proximal ring 306 via a plurality of slotted prosthetic leaflets anchors 312 having a plurality of suture eyelets to facilitate suturing of prosthetic leaflets 318 (not shown) to frame 305. In a preferred embodiment, inner ring 310 has a diameter less than that of proximal ring 306 and may also have a diameter less than that of distal ring 314.

Proximal ring 306, distal ring 314, and inner ring 310 may have a generally-circular shape, but may be other shapes such as ovals or diamonds. As illustrated, proximal ring 306 may be scallop-shaped such that proximal ring 306 extends circumferentially from prosthetic leaflets anchor 312 proximally and radially outward, then distally and radially inward toward an adjacent prosthetic leaflets anchor 312. In a preferred embodiment, the proximal ends of proximal tethers 308 are coupled to spine 301 via step 303, and extend distally and radially outward such that the distal ends of proximal tethers 308 are coupled to prosthetic leaflets anchors 312. Accordingly, this configuration may optimize prosthetic leaflets shape, e.g., permits prosthetic leaflets anchors 312 to have a longer length with less angulation between spine 301 and proximal tethers 308, thereby reducing material strains and stresses during locking of the distal, implantable portion of the support and/or during crimping of prosthetic coaptation body 300. Advantageously, the frame and ring structures are expected to maximize coaptation length while minimizing valve length and/or to create a coronary sinus-like region around the leaflet area (e.g., leaflet scallops) to maximize washout.

Inner ring 310 may extend circumferentially from prosthetic leaflets anchor 312 distally and radially outward, then proximally and radially inward toward an adjacent prosthetic leaflets anchor 312. Accordingly, inner ring 310 provides additional rigidity to proximal ring 306 and prosthetic leaflets anchors 312, which permits a reduction of overall size of frame 305, thereby reducing stress during collapse of frame 305, and improving durability of prosthetic coaptation body 300. As illustrated, distal ring 314 preferably has a sinusoidal wave shape around its circumference, such that distal tethers 316 are preferably coupled to distal ring 314 at a valley of the sinusoid. Distal ring 314 may have other oscillating shapes, which may be different in form to proximal ring 306. Thus, the proximal ends of distal tethers 316 are coupled to spine 301 via step 307, and extend distally and radially outward such that the distal ends of distal tethers 316 are coupled to the valley of distal ring 314.

As shown in FIG. 3F, the proximal ends of proximal tethers 308 may be coupled to spine 301 via step 303. Step 303 may be constructed from Nitinol and may be laser welded into place on spine 301, or alternatively, via molding or adhesive. Step 303 may have a wall thickness that is smaller, larger, or equal to the wall thickness of the cut tube from which frame 305 is formed. In addition, step 303 may include relief cuts to permit step 303 to be opened out onto spine 301 for optimal clearances for welding. Step 307 may be constructed similar to step 303.

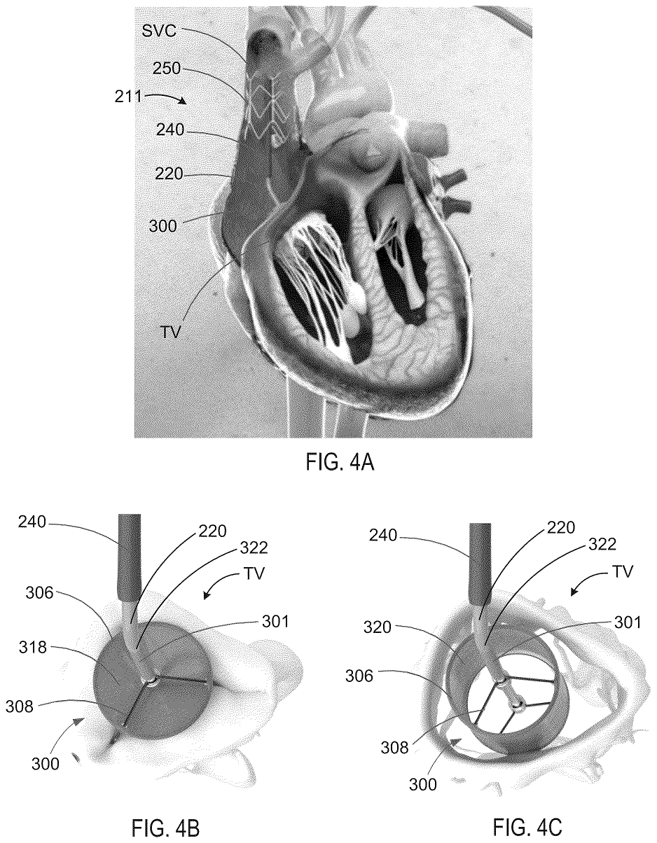

FIGS. 4A to 4C illustrate prosthetic coaptation body 300 implanted at a native heart valve. As shown in FIG. 4A, prosthetic coaptation body 300 is suspended within the native heart valve via distal, implantable portion 211 of support 200 and anchor 250. For example, anchor 250 may be implanted within SVC such that prosthetic coaptation body 300 is suspended within tricuspid valve TV via body support catheter 220, as described in further detail below. FIG. 4B shows prosthetic coaptation body 300 suspended within the tricuspid valve TV during systole whereby prosthetic leaflets 318 are in a closed configuration and the native valve leaflets are sealed against the outer surface of prosthetic coaptation body 300 to prevent regurgitation between the native leaflets and prosthetic coaptation body 300. FIG. 4C shows prosthetic coaptation body 300 suspended within the tricuspid valve TV during diastole whereby prosthetic leaflets 318 and the native valve leaflets are in an open configuration, thereby permitting flow therethrough. Advantageously, the prosthetic device sits across the native valve and contacts the native leaflets when they seal during systole, but the prosthetic device need not be in contact with the annulus. The anchoring system sits above the prosthetic device in the atrium and may extend up to the stent in the SVC or, alternatively, the IVC.

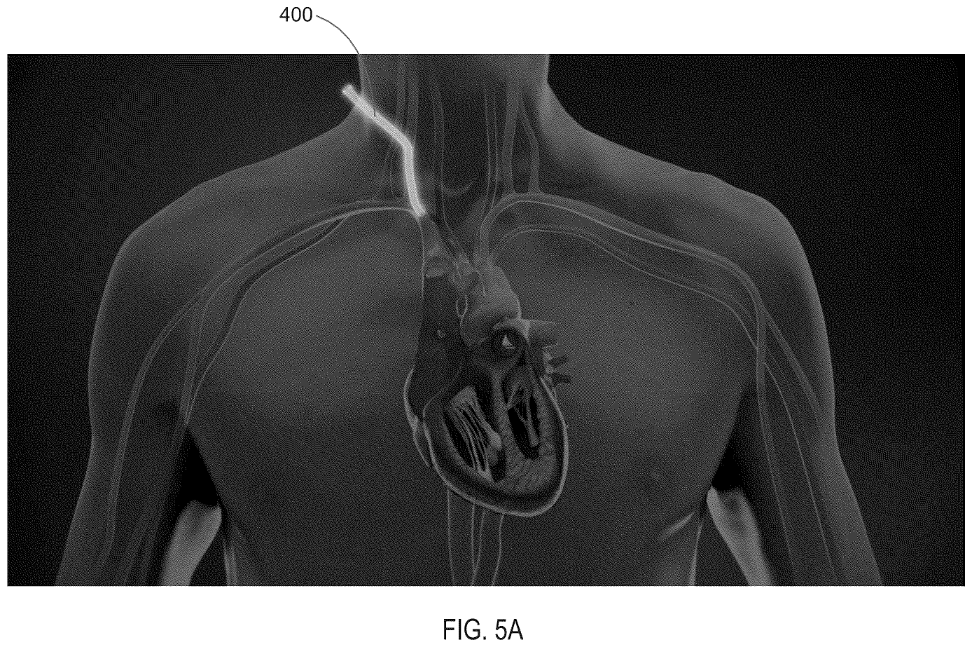

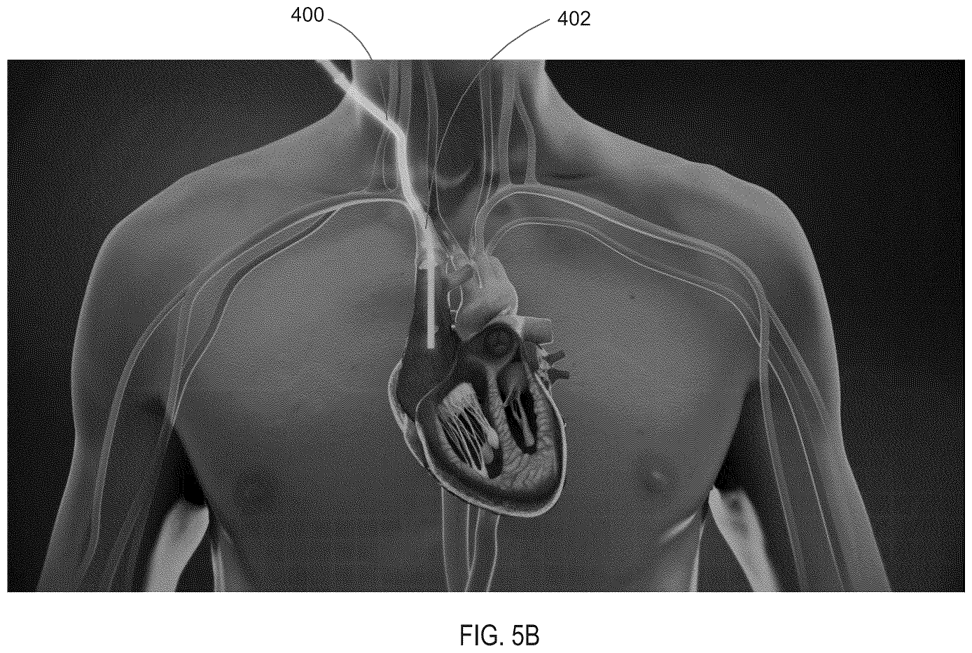

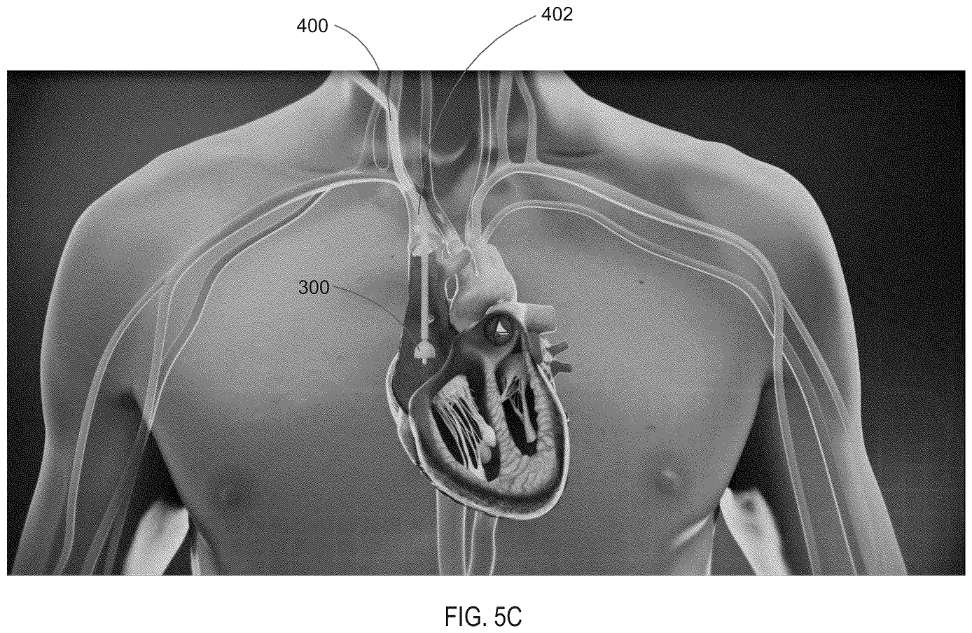

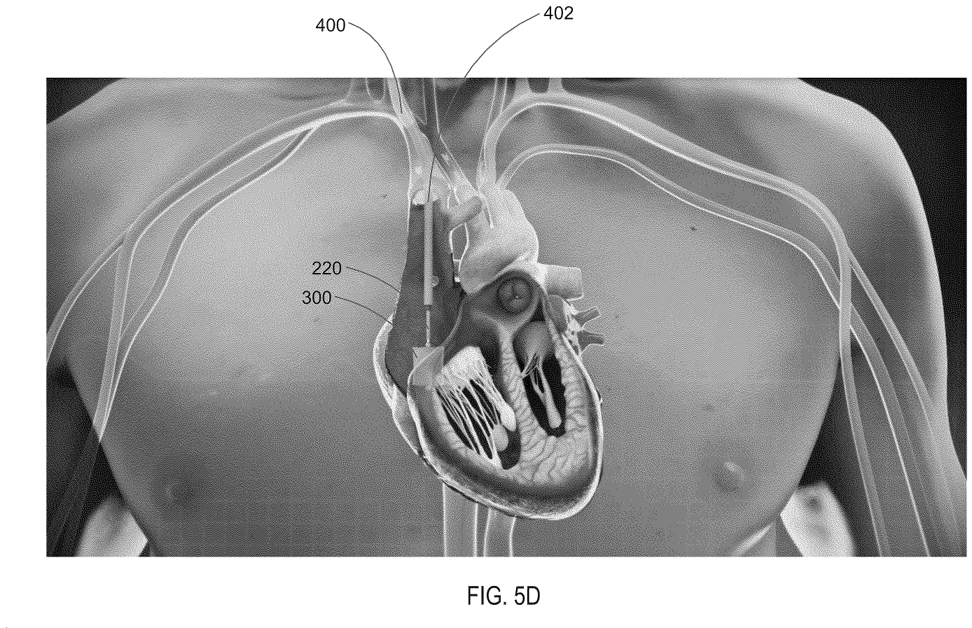

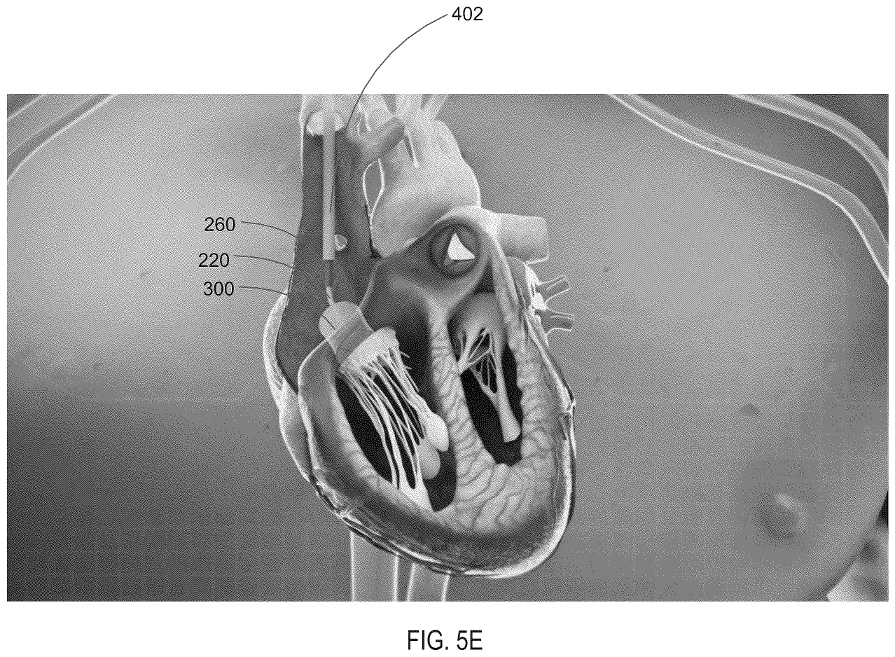

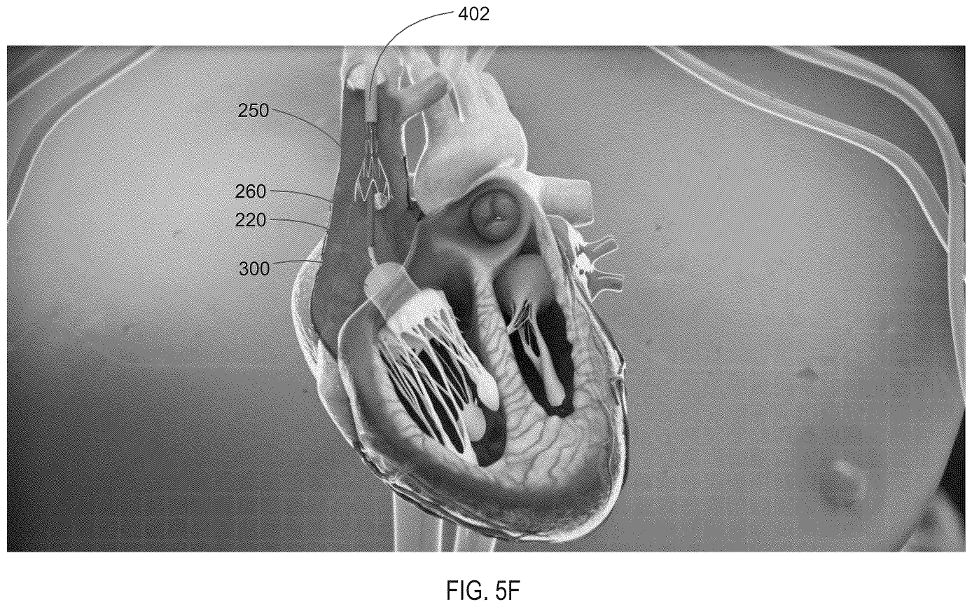

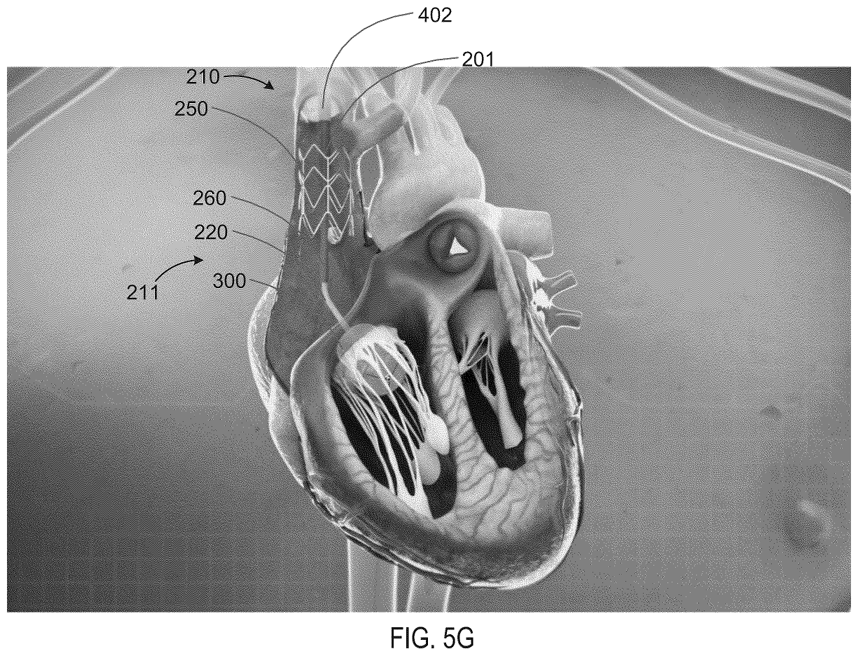

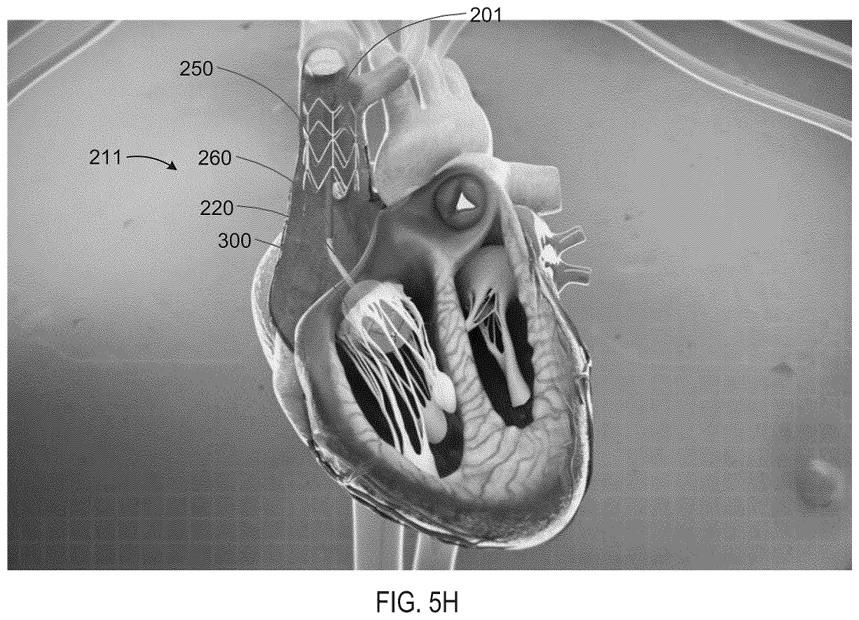

Referring now to FIGS. 5A to 5H, an exemplary method of inserting and positioning prosthetic coaptation body 300 across a native valve is shown. Prior to the implantation procedure, the patient may undergo a cardiac gated-CT to define the right heart and SVC anatomy. For the procedure, the patient may be fully anesthetized and may undergo right atrial and ventricular angiography. A sheath (e.g., 26 French sheath) may be inserted into the right internal jugular vein and a femoral venous (e.g., 9 Fr) line may be inserted for delivery of an intracardiac echo probe. All venous access may be obtained by ultrasound guidance. As shown in the figures described below, the prosthetic device may be deployed out of the delivery sheath and into the right atrium. The anchor system may then be manipulated under x-ray guidance to increase the bend angle and advance the prosthetic device until it crosses the tricuspid annulus.

Once the initial device position is achieved with the prosthetic device across the tricuspid valve, a sheath may be retracted further to deploy the stent in the SVC and then positioning may be further adjusted to determine the final, optimal position. Correct device positioning may be confirmed by fluoroscopy and echocardiography. Clinical, hemodynamic, and echocardiographic outcomes may be assessed serially during the procedure to achieve optimum position. Echocardiography may be performed at baseline and after device placement to assess device function and tricuspid regurgitation. Once the optimal position is achieved, the lock drivers in the handle may be released to lock and detach the system (e.g., a plurality of locks such as two locks and a plurality of disconnected elements such as four disconnected elements), and the handle and sheath can then be removed.

In FIG. 5A, sheath introducer 400 is inserted percutaneously into a blood vessel, e.g., through the jugular vein near a patient's neck or via the femoral artery. Sheath introducer 400 provides an opening for a clinician to percutaneously insert prosthetic coaptation body 300 and support 200 into the blood vessel. In FIG. 5B, delivery sheath 402 is inserted through sheath introducer 400 and advanced distally into the area in which prosthetic coaptation body 300 will be deployed. In the case of a damaged tricuspid valve, for example, delivery sheath 402 will extend through the inferior or superior vena cava into the right atrium such that the distal end of delivery sheath 402 is in the right atrium. In some cases, a guide wire may be introduced prior to inserting delivery sheath 402 to guide delivery sheath 402 through the blood vessel. Delivery sheath 402 preferably contains prosthetic coaptation body 300 in its compressed delivery state and support 200. Preferably, anchor 250 and anchor tube 260 may also be contained in delivery sheath 402, where anchor 250 is in a compressed delivery state.

As illustrated in FIG. 5C, prosthetic coaptation body 300 is moved distally, e.g., using actuator 108 and/or interfaces 110, 112, 114, 116, 118, 120 out the distal end of delivery sheath 402, exposing prosthetic coaptation body 300, which expands to an expanded deployed state. For example, a clinician may move actuator 108 distally while holding delivery sheath 402 in place such that the prosthetic device moves out the distal end of the sheath and self-expands upon deployment. Support 200 may alternatively be held in place while delivery sheath 402 is withdrawn to expose prosthetic coaptation body 300. FIG. 5D shows prosthetic coaptation body 300 in its fully expanded state. As illustrated in FIG. 5E, body support catheter 220 may be steered to orient prosthetic coaptation body 300 in its deployed orientation using actuator 108 and/or one or more interfaces 110, 112, 114, 116, 118, 120.

Anchor tube 260 is partially exposed, either by being pushed through delivery sheath 402 or by withdrawing delivery sheath 402 while holding in place anchor tube 260. As illustrated in FIG. 5F, delivery sheath 402 is further withdrawn, exposing anchor 250, which expands upon exposure from delivery sheath 402. As illustrated in FIG. 5G, anchor 250 is fully exposed and expanded, and engages with the walls of a blood vessel, holding itself in position. In the case of a damaged tricuspid valve, as shown, anchor 250 may engage the walls of the superior vena cava. Prosthetic coaptation body 300 may then be moved into its final deployed position using actuator 108 and/or interfaces 110, 112, 114, 116, 118, 120. For example, the interface operatively coupled to the shaping catheter may be actuated to adjust extension of the shaping catheter relative to the anchor tube to extend prosthetic coaptation body 300 to the desired distance within the native heart valve.

In addition, the interface operatively coupled to the elongated rail may be actuated to adjust the angle of the elongated rail relative to the shaping catheter such that prosthetic coaptation body 300 is a positioned at the desired angle relative to anchor 250. Moreover, the interface operatively coupled to body support catheter 220 may be actuated to telescope the body support catheter relative to the elongated rail to position prosthetic coaptation body 300 in the desired position within the native valve. Additionally, the catheters of support 200 may be rotated, e.g., by rotating actuator 108, relative to anchor 250. Once prosthetic coaptation body 300 is properly positioned, the locking and disengagement process described above may be implemented to lock the distal components of support 200 together, and disengage the proximal components of support 200 from the distal components at the detachment area so that the proximal components may be removed from the patient, as described in U.S. Pat. No. 11,219,525.

As will be understood by a person having ordinary skill in the art, when detachment of the proximal components from the distal components of the support does not require self-expanding connections, the detachment of the elongated rail, body support catheter, shaping catheter, and anchor tube may be performed independently and in any order. Because anchor tube 260 is coupled to anchor 250, prosthetic coaptation body 300 will remain in place suspended across the native valve. As illustrated in FIG. 5H, distal, implantable portion 211 remains implanted while proximal, delivery portion 210, delivery sheath 402, and sheath introducer 400 are withdrawn, e.g., by pulling actuator 108 proximally.

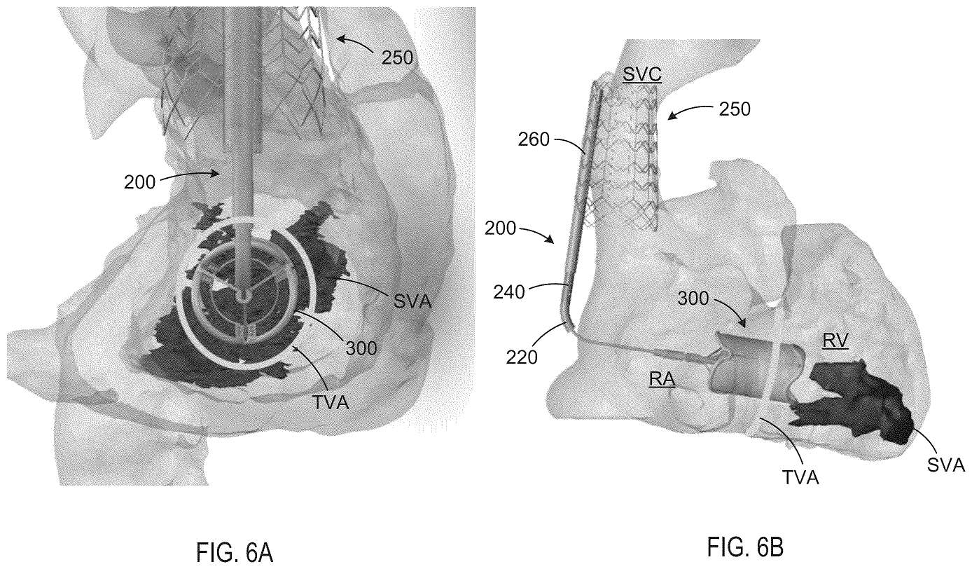

FIGS. 6A and 6B illustrate prosthetic coaptation body 300 suspended across the native heart valve, e.g., tricuspid valve annulus TVA, via support 200 and anchor 250 deployed within superior vena cava SVC. As shown in FIG. 6B, when implanted at the target location relative to tricuspid valve annulus TVA, prosthetic coaptation body 300 may be in close proximity to and/or in contact with sub-valvular apparatus SVA disposed within right ventricle RV. For example, sub-valvular apparatus SVA may comprise various structures below the level of the tricuspid valve, e.g., papillary muscles, ventricular trabeculae, chordae tendinea, etc. As described above, prosthetic coaptation body 300 is intended to effectively fill the regurgitant orifice of the native heart valve and facilitate coaptation of the native valve leaflets of the native heart valve against the skirt of prosthetic coaptation body 300, as well as against each other, during systole. However, certain physiological factors such as the complexity of the leak geometry, e.g., the geometry of the native valve leaflets, and accordingly, the regurgitant orifice, in the “closed” position during systole, may prevent the native valve leaflets from effectively coapting against prosthetic coaptation body 300 and forming an effective seal against tricuspid regurgitation. FIG. 7 illustrates various types of tricuspid valve geometries.

For example, once contact between the native valve leaflets and the skirt of prosthetic coaptation body 300 is initiated during coaptation thereof, the position of the portion of the native valve leaflets in contact with the skirt may not change, e.g., move axially in a proximal direction, relative to prosthetic coaptation body 300, which may result in the formation of additional gaps between the native valve leaflets, thereby providing additional pathways for valvular regurgitation. Moreover, when there are more than one, e.g., two, regurgitant orifices of the same/similar size, one of the regurgitant orifices may be sealed via coaptation of the corresponding leaflets against prosthetic coaptation body 300 on the corresponding side of the native valve; whereas, the other regurgitant orifice may not be sufficiently sealed via the corresponding leaflets on the corresponding side of the native valve, e.g., the leaflets do not sufficiently coapt against/mold around prosthetic coaptation body 300 adjacent the unsealed regurgitant orifice. Further, due to some leak geometries, prosthetic coaptation body 300 may not be able to completely traverse the leak gap, such that the corresponding leaflets may not effectively coapt against prosthetic coaptation body 300, leaving the regurgitant orifice unfilled for valvular regurgitation. In addition, interaction between prosthetic coaptation body 300 and sub-valvular apparatus SVA may impair function of the native valve leaflets. Accordingly, the following is a discussion of various alternative prosthetic coaptation bodies, particularly various designs of the coaptation skirt developed to enhance sealing performance while maintaining the diameter of the distal frame, e.g., distal ring 314, which may include additional reinforcement features, as described in further detail below.

Referring now to FIGS. 8A and 8B, an alternative exemplary prosthetic device of the heart valve therapeutic device having a flared distal end is provided. Prosthetic device 800 may be constructed similar to prosthetic device 300. For example, prosthetic device 800 may include frame 805 having proximal portion 802 comprising proximal ring 806 coupled to spine 801 at step 803 via proximal tethers 808 and inner ring 810 coupled to proximal ring 806 via prosthetic leaflet anchors 812, and distal portion 804 comprising distal ring 814 coupled to spine 801 at step 807 via distal tethers 816, as well as spine connector 821 and skirt 820 coupled to frame 805, which correspond to frame 305 having proximal portion 302 comprising proximal ring 306 coupled to spine 301 at step 303 via proximal tethers 308 and inner ring 310 coupled to proximal ring 306 via prosthetic leaflets anchors 312, and distal portion 304 comprising distal ring 314 coupled to spine 301 at step 307 via distal tethers 316, and spine connector 321 and skirt 320 coupled to frame 305. Distal ring 814 may have a diameter selected to prevent excessive interaction with any sub-valvular apparatus and still allow for skirt inflation during the systolic phase. In addition, prosthetic device 800 may include prosthetic leaflets (not shown) coupled to prosthetic leaflets anchors 812, e.g., via sutures, which correspond with prosthetic leaflets 318.

Prosthetic device 800 differs from prosthetic device 300 in that, while proximal end 819 of skirt 820 is coupled to proximal ring 806, e.g., via sutures, distal end 823 of skirt 820 is not coupled to distal ring 314. Moreover, the diameter of distal end 823 of skirt 820 may be larger than the diameter of distal ring 814, and accordingly, proximal ring 806, such that skirt 820 comprises a flared cone shape having a cross-sectional area that diverges from proximal end 819 towards distal end 823, as shown in FIGS. 8A and 8B. Accordingly, distal end 823 of flexible skirt 820 remains free to move independently of distal ring 814, thereby enabling skirt 820 to expand fully and conform seamlessly to the leak gap geometry of the native heart valve. Moreover, the tapering of skirt 820 at the base of the leaflets promotes blood washout and reduces risks associated with blood stasis.

In some embodiments, proximal end 819 of skirt 820 may have a larger outer diameter than the outer diameter of proximal ring 806, thereby reducing the tapered angle of skirt 820, and creating a larger volume between the inner surface of skirt 820 and the base of the prosthetic leaflets (not shown) to thereby promote blood washout at the base of the prosthetic leaflets and reduce of risks associated with blood stasis. As will be understood by a person having ordinary skill in the art, any of the prosthetic devices described herein having a skirt with a tapered profile, such that its cross-sectional area increases in the direction towards distal portion of the respective prosthetic device, may similarly have a skirt with a proximal end that is larger than the proximal portion of the frame and, thus, a smaller tapered angle, to thereby promote blood washout at the base of the leaflets of the respective prosthetic device.

Skirt 820 may be formed of a thick layer of pericardium, or multiple different layers of pericardium sutured together to create a thicker panel and, accordingly, stiffen the shape of skirt 820. The stiffness of skirt 820 (e.g., thickness of the pericardium) may be selected to allow control of the general shape of the skirt during regurgitant orifice crossing, e.g., to prevent backfolding during deploying of prosthetic device 800 and crossing of the valve annulus. As will be understood by a person having ordinary skill in the art, any of the prosthetic devices described herein may similarly be formed of multiple different layers of pericardium. The default valve shape may be controlled from the suturing pattern or alternatively, from a pericardium shape set process.

Moreover, the open distal end 823 of skirt 820 provides sufficient radial compliance to allow skirt 820 to inflate during the systolic phase and conform to the general shape of the regurgitant orifice of the native heart valve, thereby optimizing sealing of the regurgitant orifice and promoting skirt deformation towards the regurgitant commissure gaps. Further, when the prosthetic leaflets open under diastolic blood flow during the diastole phase, the inner diameter of distal end 823 being larger than the inner diameter of proximal end 819 causes skirt 820 to decelerate the velocity of blood flow along the length of the inner surface of skirt 820. As per the Bernoulli principle, the decrease in fluid velocity through device 800 results in an increase in dynamic pressure, thereby reducing collapse of the unsupported section of skirt 820 during systole. Thus, the design of skirt 820 ensures optimal adaptability by effectively adapting to the surrounding structures and achieving a superior seal to minimize or eliminate valvular regurgitation.

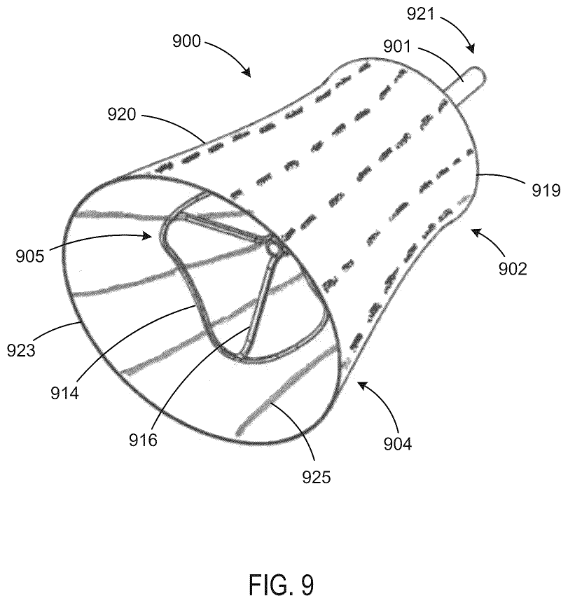

Referring now to FIG. 9, an alternative exemplary prosthetic device having a flared distal end and reinforcement skirt shaping elements is provided. Prosthetic device 900 may be constructed similar to prosthetic device 800. For example, prosthetic device 900 may include frame 905 having proximal portion 902 and distal portion 904 comprising distal ring 914 coupled to spine 901 via distal tethers 916, spine connector 921, and skirt 920 having proximal end 919 coupled to the proximal ring of frame 905 and free distal end 923. Accordingly, skirt 920 similarly comprises a flared/inverted cone shape having a cross-sectional area that diverges from proximal end 919 towards distal end 923, as shown in FIG. 9. Alternatively, in some embodiments, the degree of the angle of the cone shape of skirt 920 may be less than (e.g., flatter) that shown in FIG. 9, e.g., by increasing the outer diameter of the proximal ring of frame 905. Moreover, a proximal region of skirt 920 may comprise one or more additional skirt layers (e.g., pericardium) to thereby increase its rigidity and maintain its inflated shape. In addition, prosthetic device 900 similarly may include prosthetic leaflets (not shown) coupled to the prosthetic leaflet anchors at proximal portion 902 of frame 905.

Prosthetic device 900 differs from prosthetic device 800 in that, prosthetic device 900 may include a plurality of longitudinally extending pre-shaped skirt stiffeners, e.g., skirt stiffening elements 925, configured to control the overall shape of flared skirt 920. For example, skirt stiffening elements 925 each may have a first end coupled to proximal region 902 of frame 905, e.g., via the proximal ring of frame 905, and a second opposite end coupled to distal end 923 of skirt 920, such that skirt stiffening elements 925 extend longitudinally within skirt 920 from proximal end 919 to distal end 923 of skirt 920. As shown in FIG. 9, distal end 923 of skirt 920 may extend a predefined distance distally beyond distal ring 914 of frame 905. In some embodiments, distal end 923 of skirt 920 may extend only a minimal distance, e.g., a few millimeters, distally beyond distal ring 914 of frame 905.

Moreover, skirt stiffening elements 925 may be formed of, e.g., pre-shaped thin Nitinol wires, which may either be formed integrally with frame 905, e.g., cut from the same metal tube, or subsequently coupled thereto, e.g., via welding. Skirt stiffening elements 925 are designed to provide sufficient rigidity to prevent skirt 920 from folding backward, e.g., in the proximal direction, during native valve annulus crossing or under systolic pressure. Accordingly, skirt stiffening elements 925 help maintain distal end 923 in an open configuration, thereby promoting inflation of skirt 920 during systole. At the same time, skirt stiffening elements 925 are engineered to be compliant enough to conform to the regurgitant orifice of the native heart valve and prevent interference with the native valve leaflet commissures during coaptation.

In some embodiments, skirt stiffening elements 925 may incorporate additional longitudinal layers of pericardium or feature specific suturing patterns to achieve the desired shaping and functionality. Additionally, or alternatively, in some embodiments, skirt 920 may comprise one or more thick, longitudinal strips of pericardium sutured in a manner so as to define a hinge extending along skirt 920 between proximal end 919 and distal end 923. Accordingly, each of the one or more hinges may function as a folding apex that may be aligned with the native valve commissure to thereby permit deeper sealing of skirt 920 into the commissure.

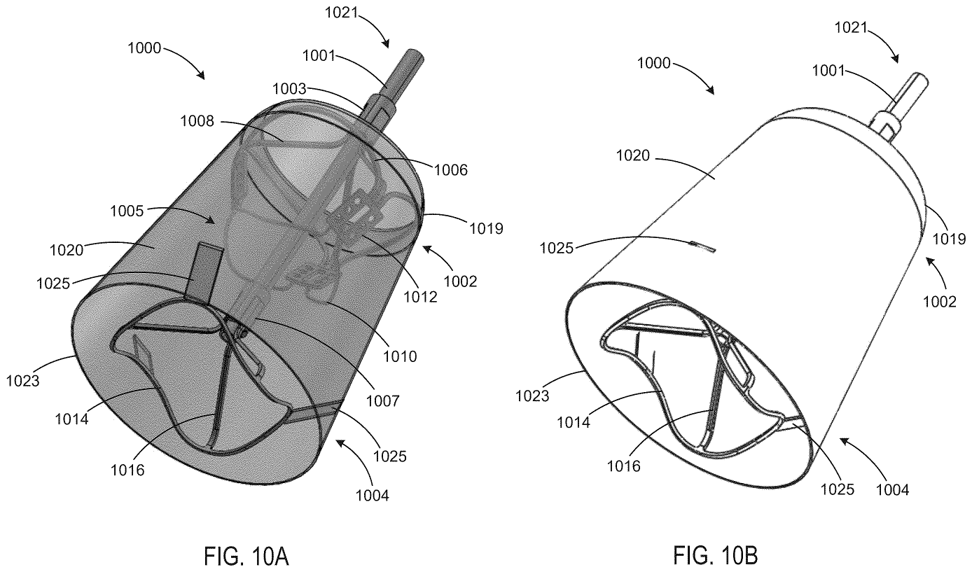

Referring now to FIGS. 10A and 10B, an alternative exemplary prosthetic device having a flared distal end and a plurality of distal ring tethers is provided. Prosthetic device 1000 may be constructed similar to prosthetic device 800. For example, prosthetic device 1000 may include frame 1005 having proximal portion 1002 comprising proximal ring 1006 coupled to spine 1001 at step 1003 via proximal tethers 1008 and inner ring 1010 coupled to proximal ring 1006 via prosthetic leaflet anchors 1012, and distal portion 1004 comprising distal ring 1014 coupled to spine 1001 at step 1007 via distal tethers 1016, as well as spine connector 1021 and skirt 1020 coupled to frame 1005. Accordingly, skirt 1020 similarly comprises a flared cone shape having a cross-sectional area that diverges from proximal end 1019 towards distal end 1023, as shown in FIGS. 10A and 10B. In addition, prosthetic device 1000 similarly may include prosthetic leaflets (not shown) coupled to prosthetic leaflet anchors 1012 at proximal portion 1002 of frame 1005.

Prosthetic device 1000 differs from prosthetic device 900 in that, prosthetic device 1000 may include a plurality of flexible skirt tethers 1025, each having a first end coupled to distal ring 1014 and a second opposite end coupled to an inner surface of skirt 1020. As shown in FIG. 10B, the second end of skirt tethers 1025 may extend at least partially through skirt 1020, such that the second end of skirt tethers 1025 may be at least partially affixed to the outer surface of skirt 1020, e.g., via additional suturing to thereby strengthen suturing. Skirt tethers 1025 are configured to provide additional support and stability to skirt 1020 to thereby maintain skirt 1020 in its intended position relative to spine 1001 and/or the native heart valve throughout the cyclic loading and mitigate the risk of skirt prolapse, e.g., prevent backfolding of distal end 1023 of skirt 1020 during crossing or inflation during systole. Skirt tethers 1025 may be made of suture material or pericardium strips sutured between the inner surface of skirt 1020 and distal ring 1014.

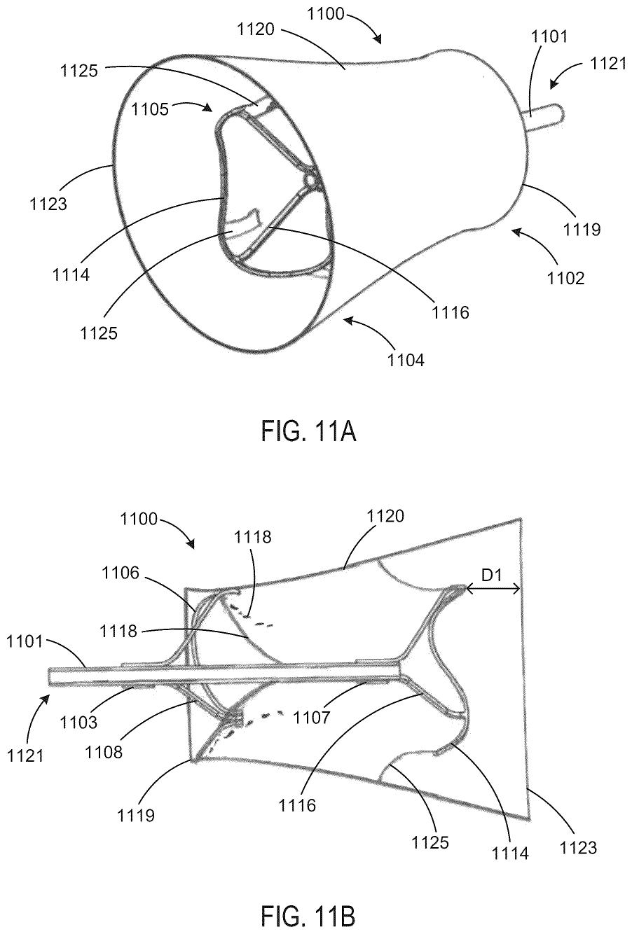

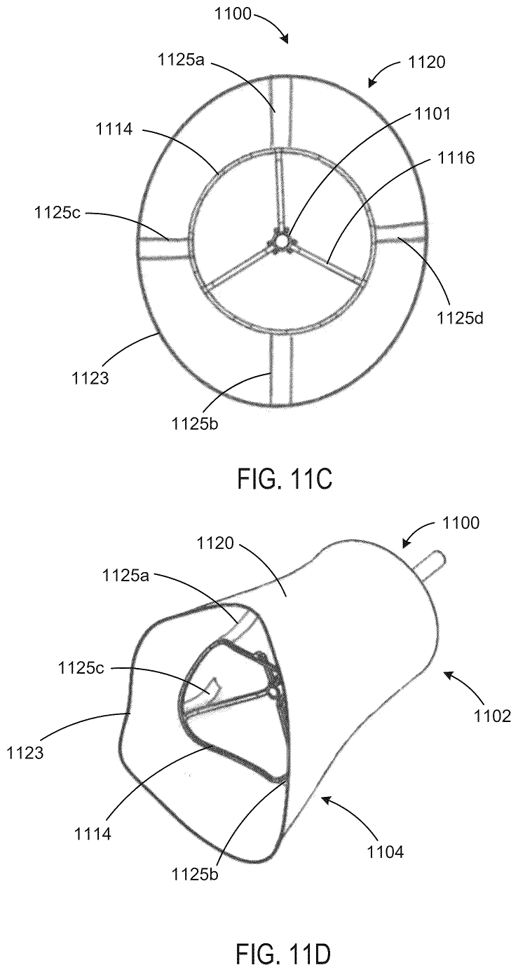

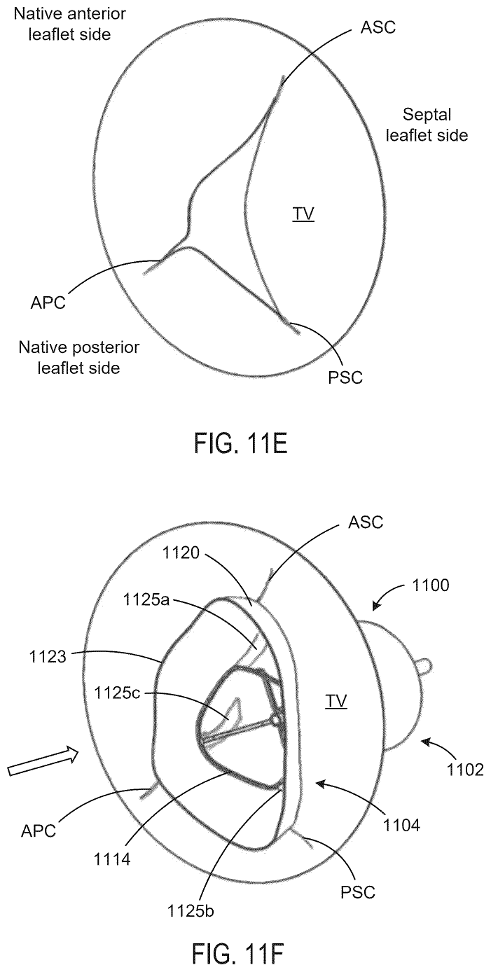

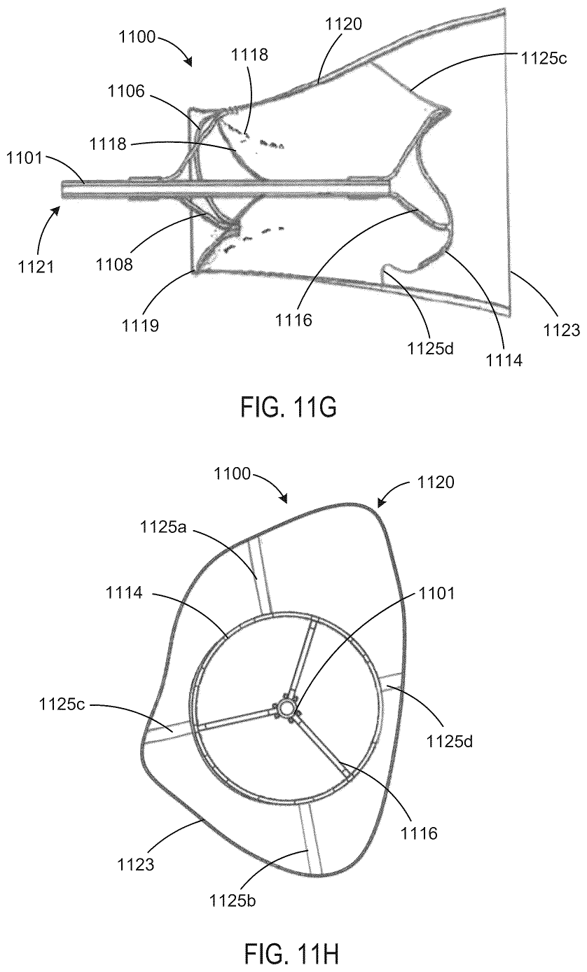

The default gap between the inner surface of skirt 1020 and frame 1005 allows for self-centering of skirt 1020 within the native heart valve post implant release. In some embodiments, each of skirt tethers 1025 may have a uniform length and be equally disposed about the circumference of distal ring 1014. Alternatively, each skirt tether may have a corresponding length selected to control a shape of the skirt, as described in further detail below with regard to FIGS. 11A to 11H. As will be understood by a person having ordinary skill in the art, while FIG. 10A illustrates skirt tethers 1025 coupled to the peaks of the sinusoidal wave shape of distal ring 1014, skirt tethers 1025 may instead be coupled to the valleys of the sinusoidal wave shape of distal ring 1014, or a combination of both peaks and valleys, e.g., based on the desired profile of skirt 1020.

Referring now to FIGS. 11A to 11H, another alternative exemplary prosthetic device having a flared distal end and a plurality of distal ring tethers is provided. Prosthetic device 1100 may be constructed similar to prosthetic device 1000. For example, prosthetic device 1100 may include frame 1105 having proximal portion 1102 comprising proximal ring 1106 coupled to spine 1101 at step 1103 via proximal tethers 1108 and an inner ring (not shown) coupled to proximal ring 1106 via prosthetic leaflet anchors (not shown), and distal portion 1104 comprising distal ring 1114 coupled to spine 1101 at step 1107 via distal tethers 1116, as well as spine connector 1121, and skirt 1120 having proximal end 1119 coupled to proximal ring 1106 of frame 1105, e.g., via sutures, and distal end 1123 coupled to distal ring 1114 via plurality of skirt tethers 1125. Accordingly, skirt 1120 similarly comprises a flared cone shape having a cross-sectional area that diverges from proximal end 1119 towards distal end 1123, as shown in FIGS. 11A and 11B. In addition, prosthetic device 1100 similarly may include prosthetic leaflets 1118 at proximal portion 1102 of frame 1105.

Prosthetic device 1100 differs from prosthetic device 1000 in that distal end 1123 of skirt 1120 may extend a predefined distance, e.g., D1, distally beyond distal ring 1114, as shown in FIG. 11B. As the coaptation distance of skirt 1120 is increased while the length of frame 1105 is minimized, distal end 1123 of skirt 1120 may be prevented from getting caught on distal ring 1114. Moreover, as shown in FIG. 11C, which illustrates device 1100 prior to coaptation, device 1110 may include four skirt tethers, e.g., 1125a, 1125b, 1125c, 1125d, each having a first end coupled to distal ring 1114 and a second opposite end coupled to skirt 1120. For example, skirt tethers 1125a and 1125b may be coupled to opposite sides of distal ring 1114, and skirt tethers 1125c and 1125d may be coupled to another pair of opposite sides of distal ring 1114. Preferably each of skirt tethers 1125a, 1125b, 1125c, 1125d may have a corresponding length selected to control a shape of skirt 1120 when inflated without radial restriction. For example, skirt tethers 1125c, 1125d may have a length that is shorter than the length of skirt tethers 1125a, 1125b, thereby causing skirt 1120 to have an oval/elliptical shaped cross-section in an inflated state, e.g., prior to coaptation, as shown in FIG. 11C. FIG. 11D illustrates the expected shape of skirt 1120 upon coaptation, e.g., in a coaptation state during the systolic phase. The length of each skirt tether may be selected to control the maximum distance that the corresponding portion of skirt 1120 is able to move radially outwardly from frame 1105. Accordingly, skirt 1120 is transitionable between an inflated state, e.g., an expanded state defined by internal forces acting on the inner surface of skirt 1120 without external coaptation forces acting on the outer surface of skirt 1120, a contracted state defined by external forces acting on the outer surface of skirt 1120 during diastole, and an expanded coaptation state defined by internal forces acting on the inner surface of skirt 1120 and external coaptation forces acting on skirt 1120 during systole.

As described above, spine connector 1121 may be rotatably fixed to the valve connector of support 200, such that the orientation/positioning of device 1100 relative to the geometry of the regurgitant orifice may be controlled during implantation. For example, as shown in FIG. 11E, the larger tricuspid regurgitant gaps generally may extend in the direction between the antero septal commissure ASC (e.g., between the native anterior leaflet side and the septal leaflet side) and the posterior septal commissure PSC (e.g., between the septal leaflet side and the native anterior leaflet side). Accordingly, the long-axis of the oval/elliptical shape of skirt 1120, e.g., formed by skirt tethers 1125a, 1125b, may be aligned towards the ASC-PSC direction, as shown in FIG. 11F, which illustrates the native leaflets of tricuspid valve TV sealed against the outer surface of skirt 1120 under tension during systole, thereby promoting a controlled shape of skirt 1120 when inflated without radial restriction. As will be understood by a person having ordinary skill in the art, FIG. 11F illustrates the contact edge of the native leaflets sealed against skirt 1120, with the portion of the native leaflets folded against skirt 1120 under tension not shown.

During systole, the blood flow direction (denoted by the arrow in FIG. 11F), closes the native leaflets of the regurgitant orifice against the flared skirt of device 1100, as well as prosthetic leaflets 1118, as shown in FIG. 11G, which illustrates prosthetic leaflets 1118 in a closed state (solid lines) during systole and an open state (dashed lines) during diastole. The closure of the native leaflets and prosthetic leaflets 1118 increases the pressure in the right ventricle which results in the inflation of skirt 1120 towards the native leaflets and towards the path of lowest resistance (e.g., within the regurgitant orifice), resulting in a shaping of the flared skirt that deforms and fills towards the native leaflet commissures. For example, based on the path of lowest resistance to fill the regurgitant orifice of the native leaflet commissures, as shown in FIGS. 11G and 11H, skirt tethers 1125a, 1125b, 1125c may be tensioned (e.g., almost fully extended) in the direction where the skirt fills the regurgitant orifices, while loosening the tension of skirt tether 1125d (e.g., folded), as shown in FIG. 11G.

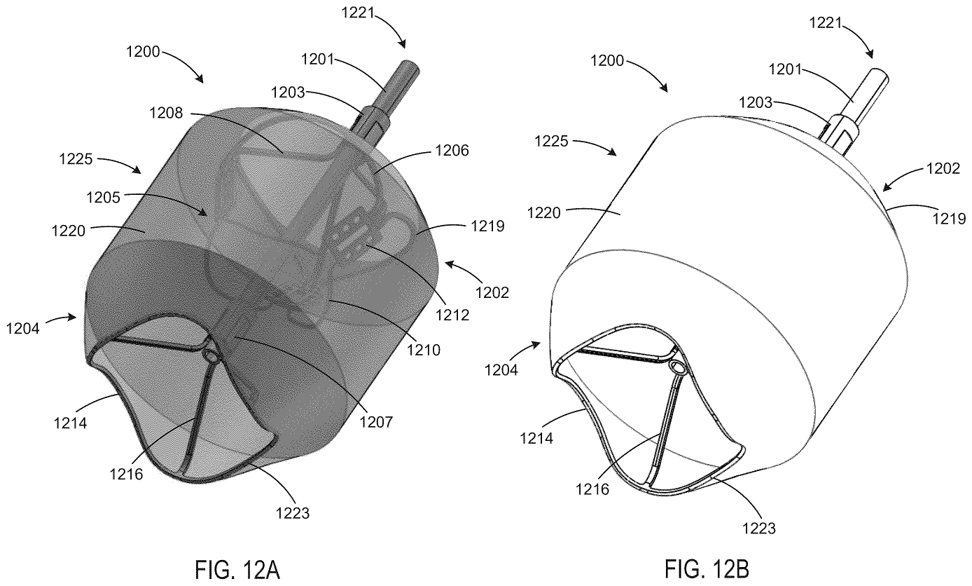

Referring now to FIGS. 12A and 12B, an alternative exemplary prosthetic device having an enlarged middle portion is provided. Prosthetic device 1200 may be constructed similar to prosthetic device 300. For example, prosthetic device 1200 may include frame 1205 having proximal portion 1202 comprising proximal ring 1206 coupled to spine 1201 at step 1203 via proximal tethers 1208 and inner ring 1210 coupled to proximal ring 1206 via prosthetic leaflet anchors 1212, and distal portion 1204 comprising distal ring 1214 coupled to spine 1201 at step 1207 via distal tethers 1216, as well as spine connector 1221, and skirt 1220 having proximal end 1219 coupled to proximal ring 1206 of frame 1205, e.g., via sutures, and distal end 1223 coupled to distal ring 1214, e.g., via sutures. In addition, prosthetic device 1200 similarly may include prosthetic leaflets (not shown) coupled to prosthetic leaflets anchors 1212, e.g., via sutures.

Prosthetic device 1200 differs from prosthetic device 300 in that device 1200 defines a larger valve than device 300. A larger valve allows for an enhanced sealing capability, providing improved functionality in the target anatomy, e.g., tricuspid valves with larger tricuspid regurgitation. Particularly, a focal increase in diameter in the middle portion of the coaptation skirt may facilitate improved structural adaptation while maintaining the distal geometry to ensure an appropriate fit in the right ventricle as well as a performant seal for smaller tricuspid regurgitation. As shown in FIGS. 12A and 12B, skirt 1220 comprises enlarged, unsupported middle portion 1225 disposed between proximal portion 1202 and distal portion 1204, wherein enlarged middle portion 1225 has a larger outer diameter than proximal end 1219 and distal end 1223, e.g., when skirt 1220 is inflated during the systolic phase. Skirt 1220 may be made from pericardium, and may be sutured to proximal ring 1206 and distal ring 1214 of frame 1205 to define a ballon type shape.

As shown in FIGS. 12A and 12B, in the inflated state, proximal portion 1202 may have a tapered profile having a cross-sectional area that increases from proximal end 1219 towards middle portion 1225, and distal portion 1204 may have a tapered profile having a cross-sectional area that increases from distal end 1223 towards middle portion 1225. Middle portion 1225 may not be attached to frame 1205, such that the looseness thereof may promote compliance during the systolic phase of the cardiac cycle to shape skirt 1220 towards the regurgitant orifice. Accordingly, enlarged middle portion 1225 of skirt 1220 inflates with compliance during systole, effectively filling regurgitant gaps and prevent regurgitation. The general shape of skirt 1220 with the increase diameter in middle portion 1225 and tapered proximal and distal portions 1202, 1204 may be controlled via suturing or skirt shape setting. For example, as shown in FIGS. 12A and 12B, middle portion 1225 may have a generally cylindrical profile between proximal portion 1202 and distal portion 1204. However, in some embodiments, middle portion 1225 may have a slightly tapered profile, such that its cross-sectional area increases in the direction towards distal portion 1204. As will be understood by a person having ordinary skill in the art, any of the prosthetic devices described herein having a skirt with an enlarged middle portion may similarly have an enlarged middle portion that is slightly tapered, such that its cross-sectional area increases in the direction towards distal portion of the respective prosthetic device, thereby promoting reduced deflation during diastole and providing an increased sealing cross-section.

Additionally, this design, e.g., the general expansion in diameter from proximal end 1219 where skirt 1220 and the prosthetic leaflets are sutured to middle portion 1225 may promote effective washout behind the prosthetic leaflets within skirt 1220 during the cardiac cycle, thereby reducing the risk of thrombus formation and preventing risks associated with blood stasis. Moreover, this geometry optimizes the interaction between device 1200 and the sub-valvular apparatus within the right ventricle.

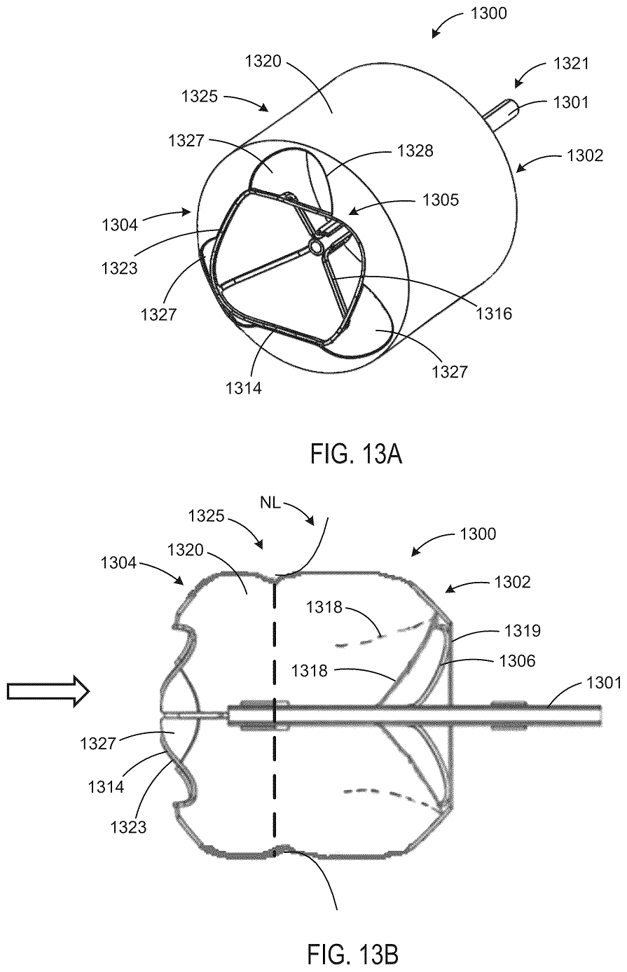

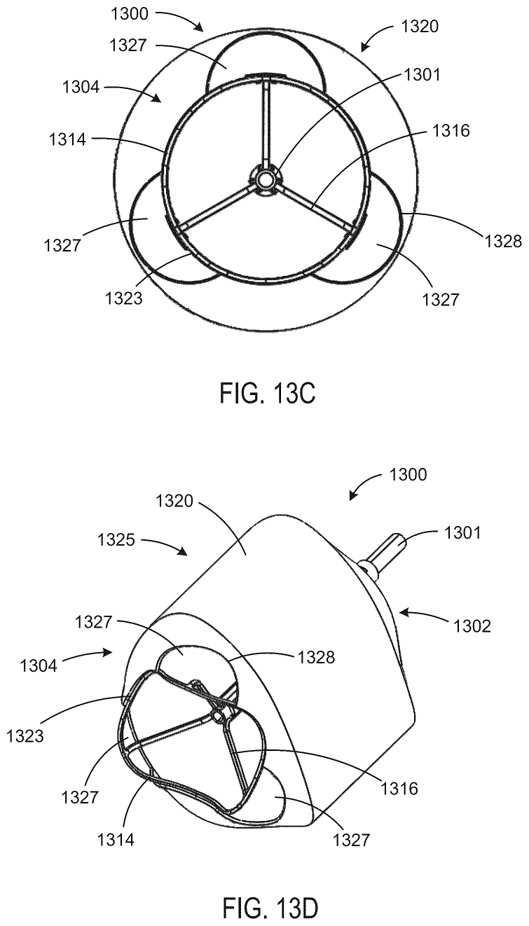

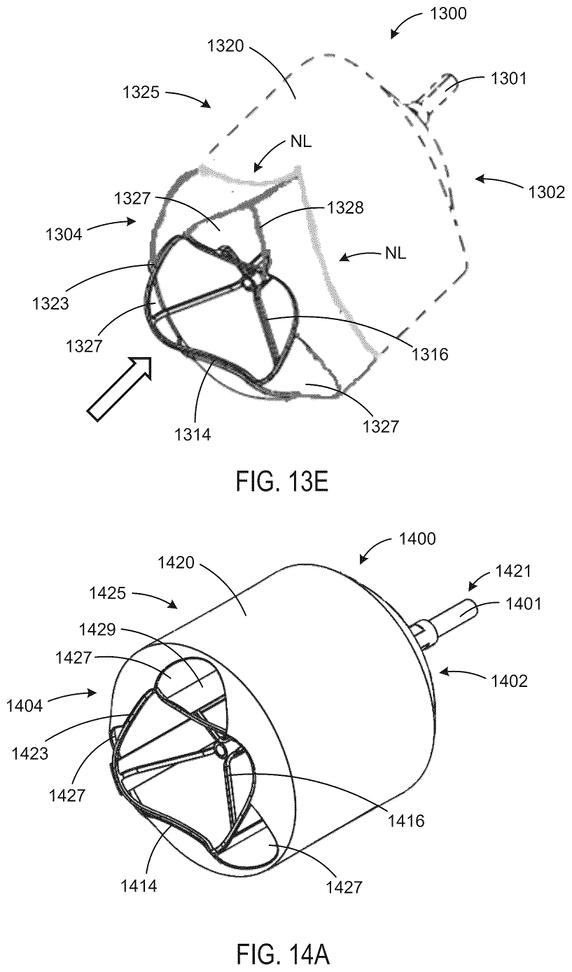

Referring now to FIGS. 13A to 13D, another alternative exemplary prosthetic device having an enlarged middle region is provided. Prosthetic device 1300 may be constructed similar to prosthetic device 1200. For example, prosthetic device 1300 may include frame 1305 having proximal portion 1302 comprising proximal ring 1306 coupled to spine 1301 via proximal tethers (not shown), and distal portion 1304 comprising distal ring 1314 coupled to spine 1301 via distal tethers 1316, as well as spine connector 1321, and skirt 1320 having proximal end 1319 coupled to proximal ring 1306 of frame 1305, e.g., via sutures, distal end 1323 coupled to distal ring 1314, e.g., via sutures, and enlarged middle portion 1325. In addition, prosthetic device 1300 similarly may include prosthetic leaflets 1318 coupled to the prosthetic leaflet anchors at proximal portion 1302 of frame 1305.

Prosthetic device 1300 differs from prosthetic device 1200 in that skirt 1320 comprises a plurality of distal windows 1327 extending at least partially along distal portion 1304, and sized and shaped to promote inflation of skirt 1320 by permitting systolic blood flow therethrough to contact the inner wall of skirt 1320 during systole. As shown in FIGS. 13A, 13D, and 13E, portions of distal end 1323 of skirt 1320 may be sutured to either the distal-most portion of frame 1305 (e.g., adjacent the valleys of the sinusoidal wave shape of distal ring 1314) such that unaffixed portions of distal end 1323 (e.g., free edges 1328) define windows 1327. This partial attachment allows for skirt 1320 to reduce in diameter partially to prevent excessive sub-valvular apparatus interaction. Moreover, this partial suturing prevents backfolding of skirt 1320 during valve deployment and valve annulus crossing. Like prosthetic device 1200, the general shape of skirt 1320 may be defined by the suturing profile, flat pattern, and/or shape setting.

During systole, the blood flow direction (denoted by the arrow in FIGS. 13B and 13E) closes the native leaflets of the regurgitant orifice against the flared skirt of device 1300, as well as prosthetic leaflets 1318, as shown in FIG. 13B, which illustrates prosthetic leaflets 1318 in a closed state (solid lines) during systole and an open state (dashed lines) during diastole. As described above, upon inflation, the looseness of enlarged middle portion 1325 will cause skirt 1320 to follow generally the path of least resistance and inflate towards the regurgitant commissure gap, as shown in FIG. 13D (without native leaflets shown) and 13E (with native leaflets NL shown), thereby providing an efficient seal, and preventing valvular regurgitation. Moreover, as shown in FIGS. 13A and 13B, middle portion 1325 may have a generally cylindrical profile between proximal portion 1302 and distal portion 1304. However, in some embodiments, middle portion 1325 may have a slightly tapered profile, such that its cross-sectional area increases in the direction towards distal portion 1304. For example, the tapered profile of middle portion 1325 further may provide support to maintain windows 1327 in an open state.

Windows 1327 provide an additional path for blood within skirt 1320 to exit the skirt during diastole. As windows 1327 are disposed radially outward from distal ring 1314, blood exiting via windows 1327 during diastole provides a mean deceleration of fluid velocity along the length of inner surface of skirt 1320 per the Bernoulli principle, which increases internal dynamic pressure, thereby reducing skirt deflation (e.g., preventing excessive collapse of the unsupported distal portions of skirt 1320 during diastole, as described above). Moreover, the slightly tapered profile of middle portion 1325 may further increase the internal pressure due to the Bernoulli principle (e.g., in combination with windows 1327). Further, during systole, the proximal region of skirt 1320 proximal to the native leaflet sealing plane (denoted by the vertical dashed line in FIG. 13B), e.g., the atrial side, inflates to an outer diameter greater than the diameter of the sealing contact between skirt 1320 and the native leaflets. The internal pressure of skirt 1320 reduces from the systolic phase to the diastolic phase. Accordingly, this alternating pattern of inflation and internal pressure reduction during the cardiac cycle generates radial motion of skirt 1320, which promotes blood washout and reduces the risks associated with blood statis. Additionally, as the atrial side of skirt 1320 inflates to an outer diameter greater than the outer diameter of the base of leaflets 1318, a larger volume is created between the inner surface of skirt 1320 and the base of leaflets 1318, thereby allowing for higher blood washout, and further reduction of risks associated with blood stasis.

Moreover, free edge 1328 of each window (e.g., unaffixed to distal ring 1314) increases compliance of skirt 1320 to provide improved compliance to the regurgitant orifice shape, as shown in FIG. 13E, which illustrates the ventricular side of skirt 1320 while skirt 1320 is inflated to fill the regurgitant commissure gap during systole. For example, free edges 1328 create a pressure relief that allows the distal region of skirt 1320 distal to the native leaflet sealing plane, e.g., the ventricular side, to conform to the sealing geometry of native leaflets NL during systole. The compliance is improved due to the very low pressure differential between the external and internal pressure at the ventricular side of skirt 1320. Additionally, as the atrial side of skirt 1320 inflates more, thereby maintaining contact of skirt 1320 with the native leaflets during systole, the internal pressure at the atrial side of skirt 1320 is greater than the atrial pressure. Accordingly, as windows 1327 provide increased conformability of skirt 1320, which may adjust the shape of the regurgitant orifice to a certain extent, less accuracy may be required for placement of prosthetic device 1300 relative to the shape of the native leaflets leak. As will be understood by a person having ordinary skill in the art, while FIGS. 13A, 13D, and 13E illustrate windows 1327 being positioned adjacent the valleys of the sinusoidal wave shape of distal ring 1314, windows 1327 may instead be positioned adjacent the peaks of the sinusoidal wave shape of distal ring 1314, or a combination of both peaks and valleys, e.g., based on the desired profile of skirt 1320. Moreover, while FIG. 13C illustrates device 1300 as having three windows 1327, skirt 1320 may define less or more than three windows.

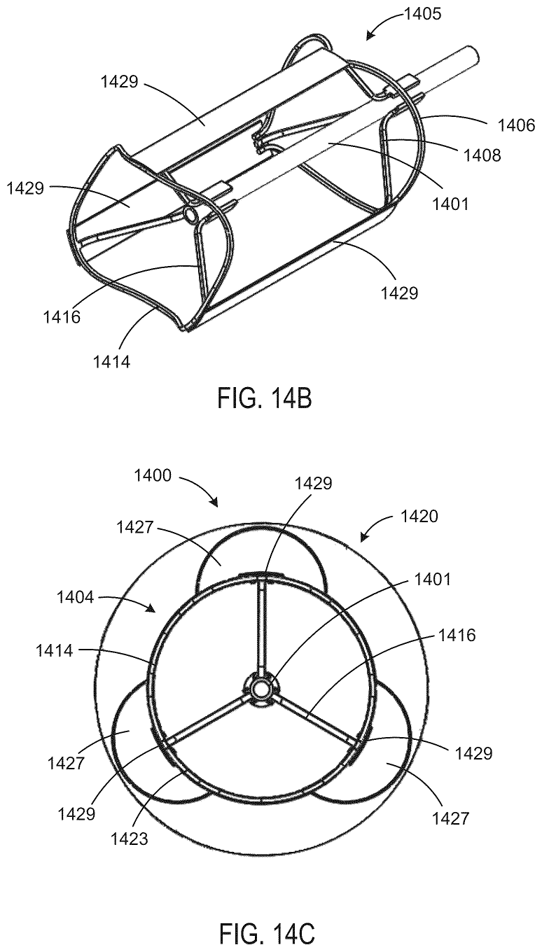

Referring now to FIGS. 14A to 14C, another alternative exemplary prosthetic device having an enlarged middle region and a plurality of distal windows is provided. Prosthetic device 1400 may be constructed similar to prosthetic device 1300. For example, prosthetic device 1400 may include frame 1405 having proximal portion 1402 comprising proximal ring 1406 coupled to spine 1401 via proximal tethers 1408, and distal portion 1404 comprising distal ring 1414 coupled to spine 1401 via distal tethers 1416, as well as spine connector 1421, and skirt 1420 having a proximal end coupled to proximal ring 1406 of frame 1405, e.g., via sutures, distal end 1423 coupled to distal ring 1414, e.g., via sutures, enlarged middle portion 1425, and a plurality of windows 1427 disposed at least partially on distal portion 1404. In addition, prosthetic device 1400 similarly may include prosthetic leaflets coupled to the prosthetic leaflet anchors (not shown) at proximal portion 1402 of frame 1405.

Prosthetic device 1400 differs from prosthetic device 1300 in that frame 1405 may include a plurality of reinforcement skirt support strips 1429 extending between proximal ring 1406 and distal ring 1414, and configured to prevent skirt collapse, e.g., during the diastolic phase of the cardiac cycle. For example, skirt support strips 1429 may be sutured under light tension at its ends to proximal ring 1406 and distal ring 1414, while having sufficient rigidity to act as a physical stop of the deflation of skirt 1420 during diastole. As described above, blood exiting via windows 1427 during diastole provides a mean deceleration of fluid velocity along the length of inner surface of skirt 1420 per the Bernoulli principle, which increases internal dynamic pressure, thereby reducing skirt deflation during diastole. Accordingly, skirt support strips 1429 may provide additional support to skirt 1420 to thereby prevent internal collapse of the unsupported distal portions of skirt 1320, e.g., windows 1427, towards spine 1401 during diastole, and preserve valve functionality.

As will be understood by a person having ordinary skill in the art, while FIG. 14B illustrates skirt support strips 1429 extending between the valleys of the sinusoidal wave shapes of proximal ring 1406 and distal ring 1414, such that skirt support strips 1429 are aligned within windows 1427, as shown in FIG. 14C, skirt support strips 1429 may extend between the peaks of the sinusoidal wave shapes of proximal ring 1406 and distal ring 1414, or a combination of both peaks and valleys, e.g., based on the desired profile of skirt 1420, and skirt support strips 1429 need not be aligned within windows 1427. Moreover, in an embodiment where the valleys of proximal ring 1406 are not aligned with the valleys of distal ring 1414, skirt support strips 1429 may extend between the aligned portions of proximal ring 1406 and distal ring 1414. Further, while FIGS. 14A to 14C illustrates device 1400 as having three skirt support strips 1429, skirt 1320 may define less or more than three skirt support strips 1429.

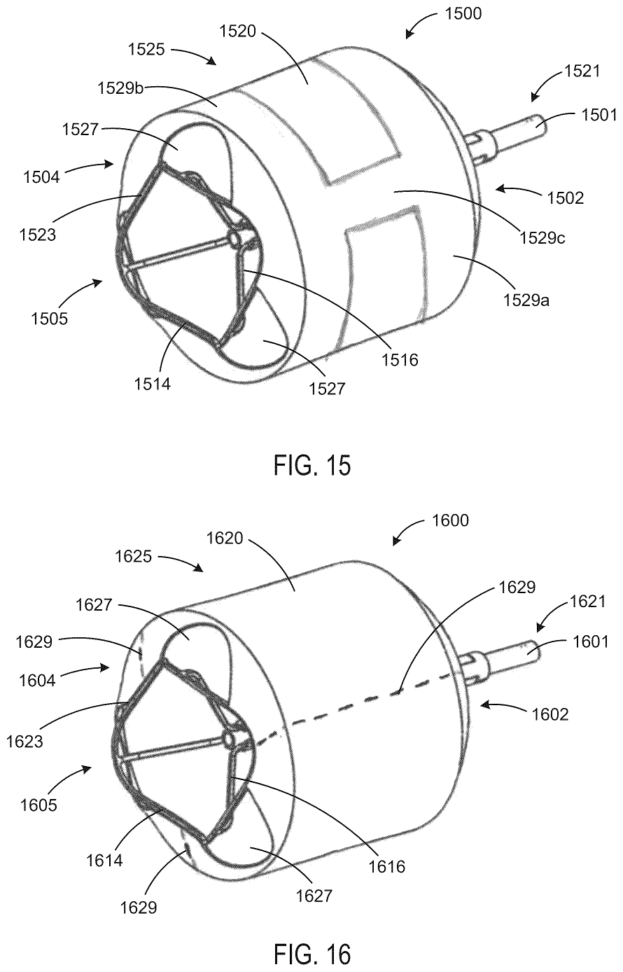

Referring now to FIG. 15, another alternative exemplary prosthetic device having an enlarged middle region and a plurality of distal windows is provided. Prosthetic device 1500 may be constructed similar to prosthetic device 1300. For example, prosthetic device 1500 may include frame 1505 having proximal portion 1502 and distal portion 1504 comprising distal ring 1514 coupled to spine 1501 via distal tethers 1516, as well as spine connector 1521, and skirt 1520 having a proximal end coupled to a proximal ring (not shown) of frame 1505, e.g., via sutures, distal end 1523 coupled to distal ring 1514, e.g., via sutures, enlarged middle portion 1525, and a plurality of windows 1527 disposed at least partially on distal portion 1504. In addition, prosthetic device 1500 similarly may include prosthetic leaflets coupled to the prosthetic leaflet anchors (not shown) at proximal portion 1502 of frame 1505.

Prosthetic device 1500 differs from prosthetic device 1300 in that skirt 1520 may include one or more skirt reinforcement support elements, e.g., proximal circumferential skirt support 1529a, distal circumferential skirt support 1529b, and/or one or more longitudinal skirt supports 1529c, configured to provide additional support to skirt 1520 and prevent skirt 1520 from backfolding over proximal portion 1502 of frame 1505, e.g., during systole. For example, as skirt 1520 inflates during the systolic phase of the cardiac cycle when the prosthetic leaflets of device 1500 close, if the enlarged middle portion is too loose, at least the proximal region of the middle portion of the skirt may backfold over the proximal portion of the frame, thereby generating a proximal pulling force on the skirt, which may reduce the coaptation diameter of the skirt, potentially impacting the efficacy of the prosthetic valve. Accordingly, proximal circumferential skirt support 1529a, distal circumferential skirt support 1529b, and/or one or more longitudinal skirt supports 1529c may provide additional rigidity locally to skirt 1520 to thereby prevent backfolding of skirt 1520 during systole. Moreover, distal circumferential skirt support 1529b further may provide additional support to maintain windows 1527 in an open state (e.g., prevent windows 1527 from collapsing internally) to ensure maximum blood flow through the distal end of prosthetic device 1500 during the cardiac cycle.

In some embodiments, proximal skirt support 1529a, distal skirt support 1529b, and longitudinal skirt support 1529c may be integrally formed from a single piece of pericardium, as shown in FIG. 15. Alternatively, in some embodiments, proximal skirt support 1529a, distal skirt support 1529b, and longitudinal skirt support 1529c may be separately formed from multiple sheets of pericardium, and sutured together at discrete regions. Proximal skirt support 1529a may be formed by multiple layers of pericardium adjacent to the proximal end of skirt 1520 and may extend entirely circumferentially along skirt 1520, to thereby provide additional rigidity along the proximal region of skirt 1520. Proximal skirt support 1529a may extend along at least the proximal region of middle portion 1525, and also may extend along proximal portion 1502. Distal skirt support 1529b similarly may be formed by multiple layers of pericardium adjacent to distal end 1523 of skirt 1520 and may extend entirely circumferentially along skirt 1520, to thereby provide additional rigidity along the distal region of skirt 1520. Distal skirt support 1529b may extend along at least the distal region of middle portion 1525, and also may extend along distal portion 1504.

Longitudinal skirt support 1529c similarly may be formed by multiple layers of pericardium, e.g., multiple strips of pericardium sutured together, and may extend between proximal skirt support 1529a and distal skirt support 1529b, to thereby provide additional rigidity (e.g., longitudinal stiffening) along the middle region of middle portion 1525 of skirt 1520. One or more longitudinal skirt supports 1529c may be disposed along the circumference of skirt 1520. The pericardium strips used to form proximal circumferential skirt support 1529a, distal circumferential skirt support 1529b, and/or one or more longitudinal skirt supports 1529c may vary in thickness, as well as in number. Moreover, the number of pericardium strips used to form longitudinal skirt supports 1529c may vary along the circumference of skirt 1520. In some embodiments, a stiffening suturing pattern may provide similar longitudinal structural stiffening of the skirt.

Referring now to FIG. 16, another alternative exemplary prosthetic device having an enlarged middle region and a plurality of distal windows is provided. Prosthetic device 1600 may be constructed similar to prosthetic device 1300. For example, prosthetic device 1600 may include frame 1605 having proximal portion 1602 and distal portion 1604 comprising distal ring 1614 coupled to spine 1601 via distal tethers 1616, as well as spine connector 1621, and skirt 1620 having a proximal end coupled to a proximal ring (not shown) of frame 1605, e.g., via sutures, distal end 1623 coupled to distal ring 1614, e.g., via sutures, enlarged middle portion 1625, and a plurality of windows 1627 disposed at least partially on distal portion 1604. In addition, prosthetic device 1600 similarly may include prosthetic leaflets coupled to the prosthetic leaflet anchors (not shown) at proximal portion 1602 of frame 1605.

Prosthetic device 1600 differs from prosthetic device 1300 in that skirt 1620 may include one or more longitudinal skirt reinforcement support elements, e.g., internal longitudinal skirt supports 1629 disposed along the circumference of skirt 1620 and configured to provide additional support to skirt 1620 and prevent skirt 1620 from backfolding over proximal portion 1602 of frame 1605, e.g., during systole, as described above. Internal longitudinal skirt supports 1629 may comprise a plurality of longitudinal semi-rigid structures sutured to skirt 1620 along its longitudinal length between the proximal end of skirt 1620 and distal end 1623. For example, internal longitudinal skirt supports 1629 may be formed of thin memory shape material extending from the proximal ring of frame 1605 and distal ring 1614, and may be pre-shaped to provide a desired shape of skirt 1620.

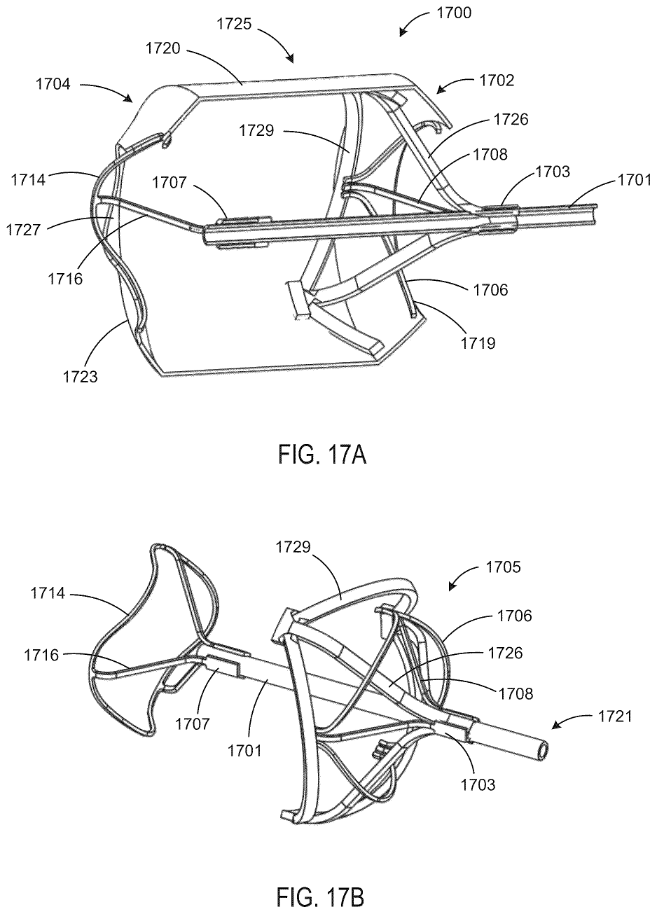

Referring now to FIGS. 17A and 17B another alternative exemplary prosthetic device having an enlarged middle region and a plurality of distal windows is provided. Prosthetic device 1700 may be constructed similar to prosthetic device 1300. For example, prosthetic device 1700 may include frame 1705 having proximal portion 1702 comprising proximal ring 1706 coupled to spine 1701 at step 1703 via proximal tethers 1708, and distal portion 1704 comprising distal ring 1714 coupled to spine 1701 at step 1707 via distal tethers 1716, as well as spine connector 1721, and skirt 1720 having proximal end 1719 coupled to proximal ring 1706 of frame 1705, e.g., via sutures, distal end 1723 coupled to distal ring 1714, e.g., via sutures, enlarged middle portion 1725, and a plurality of windows 1727 disposed at least partially on distal portion 1704. In addition, prosthetic device 1700 similarly may include prosthetic leaflets coupled to the prosthetic leaflet anchors (not shown) at proximal portion 1702 of frame 1705.

Prosthetic device 1700 differs from prosthetic device 1300 in that frame 1705 may include an additional proximal skirt reinforcement frame, e.g., secondary ring 1729 coupled to spine 1701 via secondary proximal tethers 1726 at step 1703, to provide additional support to skirt 1720 and prevent skirt 1720 from backfolding over proximal portion 1702 of frame 1705, e.g., during systole, as described above. The outer diameter of secondary ring 1729 may be equal to the outer diameter of middle portion 1725 of skirt 1720, and at least a portion of the proximal region of skirt 1720 may be coupled to secondary ring 1729, e.g., via sutures, to thereby support enlarged middle portion 1725 and prevent skirt 1720 from backfolding over proximal portion 1702 of frame 1705. As shown in FIGS. 17A and 17B, the additional proximal skirt reinforcement frame may fit over the smaller proximal frame defined by proximal ring 1706 and proximal tethers 1708. As will be understood by a person having ordinary skill in the art, prosthetic device 1700 further may include a plurality of distal windows extending at least partially along distal portion 1704 to promote inflation of skirt 1720 during systole, as described above.

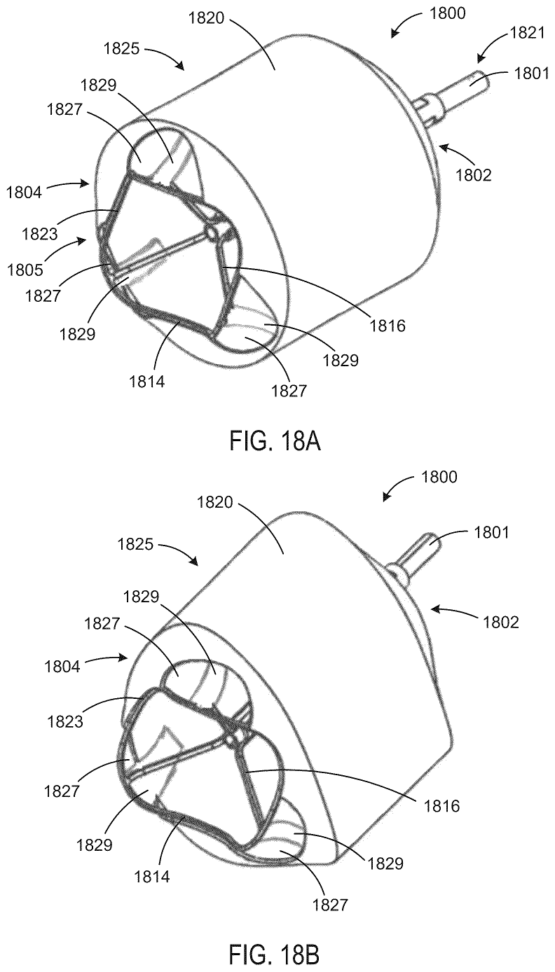

Referring now to FIGS. 18A and 18B, an alternative exemplary prosthetic device having an enlarged middle region, distal windows, and a plurality of distal ring tethers is provided. Prosthetic device 1800 may be constructed similar to prosthetic device 1300. For example, prosthetic device 1800 may include frame 1805 having proximal portion 1802 and distal portion 1804 comprising distal ring 1814 coupled to spine 1801 via distal tethers 1816, as well as spine connector 1821, and skirt 1820 having a proximal end coupled to a proximal ring (not shown) of frame 1805, e.g., via sutures, distal end 1823 coupled to distal ring 1814, e.g., via sutures, enlarged middle portion 1825, a plurality of windows 1827 disposed at least partially on distal portion 1804. In addition, prosthetic device 1800 similarly may include prosthetic leaflets coupled to the prosthetic leaflet anchors (not shown) at proximal portion 1802 of frame 1805.

Prosthetic device 1800 differs from prosthetic device 1300 in that prosthetic device 1800 may include a plurality of flexible skirt tethers 1829 configured to control the maximum deflection of skirt 1820 intended around the central portion of frame 1805, each having a first end coupled to distal ring 1814 and a second opposite end coupled to skirt 1820. Flexible skirt tethers 1829 may be constructed similar to skirt tethers 1025, 1125, described above. For example, skirt tethers 1829 may be made of suture material or pericardium strips sutured between the inner surface of skirt 1820 and distal ring 1814. Accordingly, skirt tethers 1829 may be configured to provide additional support and stability to skirt 1820 to thereby maintain skirt 1820 in its intended position relative to spine 1801 and/or the native heart valve throughout the cyclic loading and mitigate the risk of skirt prolapse, e.g., prevent backfolding of the distal region of skirt 1820 during crossing or inflation during systole.

Moreover, each of skirt tethers 1829 may have a corresponding length selected to control a shape of skirt 1820 when inflated without radial restriction, as described above. For example, skirt tethers 1829 may cause skirt 1820 to have an oval/elliptical shaped cross-section in an inflated state, e.g., prior to coaptation, as shown in FIG. 18A, as well as the expected shape illustrated in FIG. 18B upon coaptation, e.g., a coaptation state during the systolic phase. Accordingly, as spine connector 1821 may be rotatably fixed to the valve connector of support 200, the long-axis of the oval/elliptical shape of skirt 1820 may be aligned towards the ASC - PSC direction to orient/position device 1800 relative to the geometry of the regurgitant orifice, as described above.

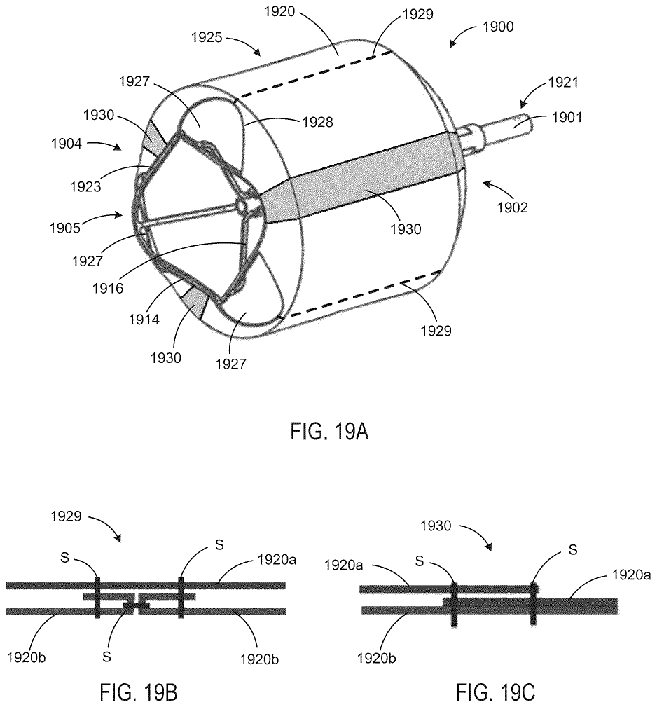

Referring now to FIGS. 19A to 19C, another alternative exemplary prosthetic device having an enlarged middle region, distal windows, and a plurality of skirt shape support panels is provided. Prosthetic device 1900 may be constructed similar to prosthetic device 1300. For example, prosthetic device 1900 may include frame 1905 having proximal portion 1902 and distal portion 1904 comprising distal ring 1914 coupled to spine 1901 via distal tethers 1916, as well as spine connector 1921, and skirt 1920 having a proximal end coupled to a proximal ring (not shown) of frame 1905, e.g., via sutures, distal end 1923 coupled to distal ring 1914, e.g., via sutures, enlarged middle portion 1925, and a plurality of windows 1927 having free edges 1928 and disposed at least partially on distal portion 1904. In addition, prosthetic device 1900 similarly may include prosthetic leaflets coupled to the prosthetic leaflet anchors (not shown) at proximal portion 1902 of frame 1905.

Prosthetic device 1900 differs from prosthetic device 1300 in that skirt 1920 may be formed of at least two layers of pericardium sutured together in a manner to define at least one skirt shape support zones, e.g., support zones 1929 and/or support zones 1930, disposed along the circumference of skirt 1920 and extending between proximal portion 1902 and distal portion 1904 at discrete regions. For example, each of support zones 1929 and support zones 1930 may be configured to provide additional support to skirt 1920 to thereby maintain the desired shape of skirt 1920 while preventing excessive proximal folding movement of skirt 1920 during systole (e.g., preventing skirt 1920 from backfolding over proximal portion 1902 of frame 1905) and excessive deflation of skirt 1920 (e.g., into the inner diameter of prosthetic device 1900 during diastole, as described above.

Each layer of pericardium may be formed by at least two panels of pericardium, thereby requiring smaller panel sizes for manufacturing efficiency. For example, the inner layer of skirt 1920 may be formed by a plurality of panels 1920b, each extending between proximal portion 1902 and distal portion 1904, and positioned adjacent one another and sutured together in a folded manner as shown in FIG. 19B. As shown in FIG. 19B, support zones 1929 may each be formed by suturing folded edges of adjacent inner panels 1920b (e.g., via sutures S) along a longitudinal length of skirt 1920 (e.g., along at least enlarged middle portion 1925), and suturing the folded portions of adjacent inner panels 1920b to outer panel 1920a of the upper layer of skirt 1920. Preferably, the edges of adjacent inner panels 1920b are folded towards outer panel 1920a, as shown in FIG. 19B.

Moreover, the upper layer of skirt 1920 may be formed by a plurality of panels 1920a, each extending between proximal portion 1902 and distal portion 1904, and positioned adjacent one another in an overlapping manner as shown in FIG. 19C. For example, as shown in FIG. 19C, support zones 1930 may each be formed by suturing overlapping portions of adjacent outer panels 1920a (e.g., via sutures S) to inner panel 1920b of the inner layer of skirt 1920 along a longitudinal length of skirt 1920 (e.g., along at least enlarged middle portion 1925). By having a minimum of two layers of pericardium, all surfaces of skirt 1920 exposed to blood flow may comprise the smooth pericardium side, thereby reducing the shear stress on blood at high flow velocities. In FIG. 19A, skirt 1920 is formed by an upper layer comprising three upper panels 1920a, and an inner layer comprising three inner panels 1920b, thereby defining three support zones 1929 and three support zones 1930. In some embodiments, to avoid a negative impact of suturing additional layers of pericardium onto the leaflets base which could potentially impair the leaflet dynamic motion, the region of skirt 1920 just radially outside the proximal ring of frame 1905 may comprise only a single layer of pericardium.

Windows 1927 may be sized and shaped to provide a maximum length of free edges 1928 to thereby maximize compliance, and the ratio of the width of windows 1927 and the length of free edges 1928 of windows 1927 may be selected such that, when the load applied by the native leaflets on the outer surface of skirt 1920 during systole (e.g., at the distal region of skirt 1920) is released, windows 1927 do not fold in/collapse. As will be understood by a person having ordinary skill in the art, the skirt design may be optimized in different embodiments to increase the number, size, shape, and/or orientation of windows 1927 (in combination with frame 1905) to increase compliance further. Moreover, the number of panels of each layer of skirt 1920 may be selected to accommodate a corresponding number of windows, e.g., to increase the number of windows, and the overlapping structure of the respective support zones may be constructed differently than that shown in FIGS. 19B and 19C.

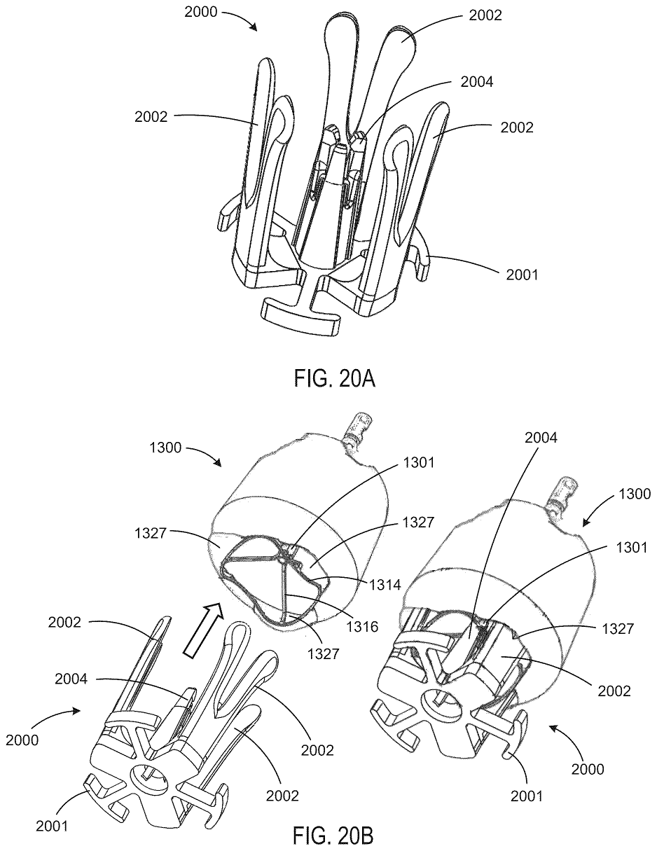

Referring now to FIGS. 20A and 20B, an exemplary prosthetic device holder for supporting the prosthetic device prior to use to preserve valve performance is provided. For example, upon assembly and prior to use of the prosthetic devices described herein, which are formed of pericardium, each prosthetic device is typically stored in a container, e.g., a jar, with a solution, such as a Glutaraldehyde solution, in its expanded/inflated configuration. As the skirt of at least some of the prosthetic device embodiments described herein (e.g., prosthetic devices 1300, 1400, 1500, 1600, 1700, 1800, 1900) comprise large portions that are unsupported by a rigid frame, prosthetic device holder 2000 may be provided to reduce/prevent risks of unwanted folding/creasing of the unsupported portions of the skirt when the prosthetic device is stored in the container, which may potentially set in shape and impact the valve performance of the prosthetic device. Accordingly, prosthetic device holder 2000 may be configured to support the shape of the skirt of the prosthetic device in the expanded/inflated configuration while the prosthetic device is stored prior to use.

For example, as shown in FIG. 20A, when the prosthetic device to be stored comprises a plurality of windows, as described above, prosthetic device holder 2000 may comprise a number of window-supporting prongs 2002 corresponding to the number of windows. Prongs 2002 may be coupled to and extend laterally from base 2001, and may be sized and shaped to be received within an interior of the skirt of the prosthetic device via a corresponding window, e.g., windows 1327 of prosthetic device 1300, as shown in FIG. 20B. When disposed within the interior of the skirt of the prosthetic device, prongs 2002 may be configured to support the windows and the skirt shape of the prosthetic device in its expanded/inflated configuration. Moreover, as shown in FIGS. 20A and 20B, each prong 2002 may comprise a pair of expandable fingers, which may be biased in an expanded state, and pinched together, e.g., by the user, towards a collapsed state for insertion through the window of the skirt. Accordingly, when the prongs are positioned within the interior of the skirt, they may return to their expanded state to provide internal support to the corresponding window and maintain the inflated skirt shape during storage, as shown in FIG. 20B. Additionally, the proximal tips of each prongs 2002 further may support the proximal region of skirt 1320, to thereby prevent creasing.

In addition, prosthetic device holder 2000 may comprise valve spine holder 2004 configured to support the spine of the prosthetic device and control the position/orientation of the prosthetic device within the container during storage. For example, as shown in FIG. 20A, valve spine holder 2004 may comprise a plurality of extensions (e.g., prongs) coupled to and extending laterally from base 2001, and defining a lumen sized and shaped for receiving the spine of the prosthetic device, e.g., spine 1301 of prosthetic device 1300, as shown in FIG. 20B. The plurality of extensions may be biased towards a radially inward collapsed state to thereby apply a force against the spine when the spine is received within the lumen defined by the plurality of extensions to thereby stabilize the spine relative to valve spine holder 2004. The number of extensions of valve spine holder 2004 may correspond with the number of tethers supporting the distal ring of the prosthetic device, e.g., tethers 1316 coupled to distal ring 1314, such that each extension of the plurality of extensions of valve spine holder 2004 may be received between adjacent tethers, and advanced longitudinally over the valve spine. Moreover, the resting surface of valve spine holder 2004 (e.g., in the axial direction) allows the user to control the depth of insertion of prong 2002 of prosthetic device holder 2000 into prosthetic device 1300 to ensure support on the full length of skirt 1320 from window 1327 to the proximal end of prosthetic device 1300, and prevent axial tensioning at proximal end of skirt 1320.

In some embodiments, base 2001 may comprise an arcuate shape that corresponds to the cylindrical/tubular shape of the storage container, such that the outer surface of base 2001 may abut the inner surface of the container during storage, thereby preventing or minimizing relative movement between prosthetic device holder 2000 (and, accordingly, the prosthetic device) and the container during storage. As will be understood by a person having ordinary skill in the art, a prosthetic device holder may similarly be used to support window-less prosthetic devices described herein having skirts that comprise large portions that are unsupported by a rigid frame, e.g., prosthetic devices 800, 900, 1000, 1100, 1200). Accordingly, such prosthetic device holders may be constructed similar to prosthetic device holder 2000 and include a valve spine holder configured to receive and support the spine of the prosthetic device, but without window-supporting prongs, as described above.

The invention is not limited to the embodiments described but may be varied in construction and detail. For example, the order in which the elongated rail, the body support catheter, and the shaping catheter are disposed within each other to form the support may vary. The described embodiments are to be considered in all respects only as illustrative and not restrictive. Any part of the device may be of a material that is visible to equipment such as echo or x-ray imaging equipment. The device may further comprise a controller arranged to be implanted subcutaneously on the support to allow the position of the prosthetic coaptation body to be changed after insertion. Electromagnetic switches may be used to activate to alter the position of the distal end of the support.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed:

1. A coaptation assist valve device for placement across a patient's native heart valve having a regurgitant orifice, the coaptation assist valve device comprising:
a frame comprising a proximal structure, a distal structure, and a spine extending between the proximal and distal structures;
a conduit comprising a proximal end coupled to the proximal structure, a distal end, and a flexible side wall defining a channel extending between the proximal and distal ends, the flexible side wall configured to transition between an inflated state, a contracted state, and an expanded coaptation state; and
a plurality of prosthetic leaflets mounted within the channel of the conduit,
wherein at least a middle portion of the flexible side wall has an outer diameter that is larger than an outer diameter of the proximal end in the inflated state, an external surface of the middle portion of the flexible side wall configured to fill the regurgitant orifice and coapt with native leaflets of the native heart valve in the expanded coaptation state.

2. The coaptation assist valve device of claim 1, wherein the plurality of prosthetic leaflets are configured to allow diastolic blood flow therethrough, while preventing systolic blood flow therethrough,
wherein the plurality of prosthetic leaflets are coupled to the proximal structure, and
wherein the plurality of prosthetic leaflets are formed from a same material as the flexible side wall.

3. The coaptation assist valve device of claim 1, wherein the proximal structure comprises a plurality of prosthetic leaflet anchors configured to be coupled to the plurality of prosthetic leaflets, the plurality of prosthetic leaflet anchors configured to couple the proximal structure to an inner structure of the frame, the inner structure configured to provide additional rigidity to the proximal structure,
wherein the proximal structure and/or the distal structure comprises a ring, the ring comprising a scallop, sinusoidal, or zig-zag shape,
wherein the proximal structure is coupled to the spine via a plurality of proximal spokes, and
wherein the distal structure is coupled to the spine via a plurality of distal spokes.

4. The coaptation assist valve device of claim 1, wherein the distal end of the conduit is not coupled to the distal structure, and wherein, in the inflated state, the distal end of the conduit has an outer diameter that is larger than an outer diameter of the distal structure.

5. The coaptation assist valve device of claim 4, wherein the flexible side wall is unsupported between the proximal and distal ends of the conduit.

6. The coaptation assist valve device of claim 4, wherein, in the inflated state, at least a portion of the flexible side wall has an outer diameter that increases in a direction from the proximal end towards the distal end, such that the flexible side wall comprises a cone shape configured to reduce the fluid velocity and increase internal dynamic pressure of the conduit during diastole, to thereby reduce deflation of the flexible side wall.

7. The coaptation assist valve device of claim 4, wherein the conduit comprises a flexibility that varies along a length of the conduit, the flexibility configured to increase compliance of the conduit in a radial direction and increase stiffness of the conduit in an axial direction to prevent the conduit from prolapsing.

8. The coaptation assist valve device of claim 7, wherein the conduit comprises a plurality of stiffeners extending longitudinally between the proximal and distal ends of the conduit within the flexible side wall, the plurality of stiffeners comprising a shape memory material pre-shaped to control an overall shape of the conduit.

9. The coaptation assist valve device of claim 7, wherein the flexible side wall comprises a thickness that that varies along the length of the conduit.

10. The coaptation assist valve device of claim 9, wherein the varying thickness of the flexible side wall is defined by a number of layers of sheet material forming the flexible side wall along the length of the conduit.

11. The coaptation assist valve device of claim 10, wherein a proximal region of the conduit comprises a plurality of layers of sheet material configured to increase rigidity and maintain the shape of the proximal region of the conduit.

12. The coaptation assist valve device of claim 10, wherein the flexible side wall comprises an outer layer of sheet material comprising a plurality of outer panels, and an inner layer of sheet material comprising a plurality of inner panels, the plurality of outer panels and inner panels sutured together to define a plurality of longitudinally extending support zones disposed along a circumference of the flexible side wall, the plurality of longitudinally extending support zones configured to increase axial rigidity of the conduit and expose blood flow across the flexible side wall to a smooth side of sheet material to reduce shear stress on the blood.

13. The coaptation assist valve device of claim 12, wherein a first longitudinally extending support zone of the plurality of longitudinally extending support zones comprises a first outer panel of the plurality of outer panels of the outer layer sutured to folded portions of adjacent inner panels of the plurality of inner panels of the inner layer, the adjacent inner panels sutured together.

14. The coaptation assist valve device of claim 12, wherein a second longitudinally extending support zone of the plurality of longitudinally extending support zones comprises a first inner panel of the plurality of inner panels of the inner layer sutured to overlapping portions of adjacent outer panels of the plurality of outer panels of the outer layer.

15. The coaptation assist valve device of claim 7, wherein the flexible side wall is pre-formed to comprise weak portions along the length of the conduit to vary the flexibility of the conduit along the length of the conduit.

16. The coaptation assist valve device of claim 1, wherein the distal end of the conduit is coupled to the distal structure, and wherein, in the inflated state, a proximal portion of the conduit has a cross-sectional area that increases from the proximal end towards the middle portion, and a distal portion of the conduit has a cross-sectional area that increases from the distal end towards the middle portion.

17. The coaptation assist valve device of claim 16, wherein the flexible side wall defines a plurality of windows disposed on at least the distal portion of the conduit, the plurality of windows sized and shaped to permit blood flow therethrough to promote inflation of the flexible side wall during systole,
wherein unsupported edges of the plurality of windows are configured to facilitate compliance of the flexible side wall to the regurgitant orifice, and
wherein blood flow through the plurality of windows is configured to reduce the fluid velocity and increase internal dynamic pressure of the conduit during diastole, to thereby reduce deflation of the flexible side wall.

18. The coaptation assist valve device of claim 17, wherein, in the inflated state, the middle portion of the conduit has an outer diameter that increases in a direction from the proximal end towards the distal end.

19. The coaptation assist valve device of claim 17, wherein, during systole, an atrial side of the conduit proximal to a native leaflet sealing plane of the conduit is configured to inflate to an outer diameter greater than a sealing contact between the conduit and the native leaflets at the native leaflet sealing plane.

20. The coaptation assist valve device of claim 19, wherein an internal pressure of the conduit decreases from systole to diastole, such that, during the patient's cardiac cycle, radial motion of the conduit is generated to promote blood washout and reduce risks associated with blood stasis.

21. The coaptation assist valve device of claim 17, wherein a ratio of a width of the plurality of windows and a length of the unsupported edges of the plurality of windows is selected to prevent the plurality of windows from folding upon release of an external force on the distal portion of the conduit.

22. The coaptation assist valve device of claim 16, wherein, in the inflated state, an inner surface of the proximal portion of the conduit is separated from the proximal structure coupled to the plurality of prosthetic leaflets by a gap, the gap configured to promote blood washout and reduce risks associated with blood stasis.

23. The coaptation assist valve device of claim 16, wherein the flexible side wall is unsupported between the proximal and distal ends of the conduit.

24. The coaptation assist valve device of claim 16, wherein the conduit comprises a proximal support extending circumferentially along at least a proximal region of the flexible side wall, a distal support extending circumferentially along at least a distal region of the flexible side wall, and/or one or more longitudinal supports extending longitudinally between the proximal and distal supports, the proximal, distal, and/or longitudinal supports configured to prevent prolapse of the flexible side wall over the frame.

25. The coaptation assist valve device of claim 1, further comprising a support coupled to the spine, the support configured to suspend the conduit across the native heart valve without anchoring of the coaptation assist valve device to an annulus of the native heart valve or atrial or ventricular tissue adjacent to the native heart valve.

26. The coaptation assist valve device of claim 25, wherein the support comprises a stent configured to engage the wall of a blood vessel, the stent configured to retain the support in a position in the patient to maintain the conduit in position across the native heart valve.

27. A holder device for supporting a coaptation assist valve device during storage within a container, the coaptation assist valve device comprising a proximal structure, a distal structure, a spine extending between the proximal and distal structures, and a flexible conduit having a proximal end coupled to the proximal structure and an at least partially unsupported distal end, the holder device comprising:
a base sized and shape to minimize movement between the holder device and the container when the holder device is disposed within the container; and
a spine support comprising a plurality of extensions extending laterally from the base, the plurality of extensions configured to be inserted through the distal structure and defining a lumen configured to receive the spine of the coaptation assist valve device,
wherein, when the spine of the coaptation assist valve device is received within the lumen and the holder device is disposed within the container, the holder device is configured to support the shape of flexible conduit in an expanded state to thereby prevent folding of the at least partially unsupported distal end of the flexible conduit.

28. The holder device of claim 27, wherein at least a portion of the distal end of the flexible conduit is coupled to the distal structure of the coaptation assist valve device to define a plurality of windows, and wherein the holder device further comprises:
a plurality of prongs extending laterally from the base, the plurality of prongs sized and shaped to be inserted through the plurality of windows,
wherein, when the plurality of prongs are disposed within the flexible conduit via the plurality of windows and the holder device is disposed within the container, the plurality of prongs are configured to support the plurality of windows and the flexible conduit in the expanded state.

29. The holder device of claim 28, wherein each of the plurality of prongs comprises a pair of fingers, the pair of fingers configured to transition between a collapsed configuration where the pair of fingers are sized and shaped to be inserted through the plurality of windows, and an expanded configuration where the pair of fingers are configured to support the flexible conduit in the expanded state, and
wherein the pair of fingers are biased towards the expanded state.

30. The holder device of claim 28, wherein the plurality of prongs comprise a number of prongs corresponding with a number of windows of the plurality of windows of the coaptation assist valve device.
